(19) United States
(12) Patent Application Publication
(10) Pub. No.: US 2018/0369334 A1
(43) Pub. Date: Dec. 27, 2018

(54) VEGF VARIANT POLYPEPTIDE COMPOSITIONS

(71) Applicant: The Board of Trustees of the Leland Stanford Junior University, Stanford, CA (US)

(72) Inventors: Jennifer R. Cochran, Stanford, CA (US); Shiven Kapur, Palo Alto, CA (US); Mark S. Blumenkranz, Portola Valley, CA (US)

(73) Assignee: The Board of Trustees of the Leland Stanford Junior University, Stanford, CA (US)

(21) Appl. No.: 15/540,216

(22) PCT Filed: Jan. 15, 2016
(86) PCT No.: PCT/US2016/013688

§ 371 Date: Jun. 27, 2017

Related U.S. Application Data
(60) Provisional application No. 62/104,590, filed on Jan. 16, 2015.
(60) Provisional application No. 62/104,588, filed on Jan. 16, 2015.
(60) Provisional application No. 62/104,621, filed on Jan. 16, 2015.

(51) Int. Cl. A61K 38/18; C07K 14/515; C07K 14/52; A61P 27/02

(52) U.S. Cl. CPC A61K 38/1866; A61P 27/02; C07K 14/52; C07K 14/515

(57) ABSTRACT

Provided herein are VEGF variant polypeptides and Fc-VEGF variant polypeptide fusions, comprising a first VEGF monomer joined to a second VEGF monomer by a peptide linker or a disulfide bridge. In some embodiments, the VEGF variant polypeptides comprise the formula: A-L-B, wherein A is a first VEGF monomer subunit; B is a second VEGF monomer subunit; and L is a peptide linker having 14 to 20 amino acids. In certain embodiments, disclosed herein, are methods of treating an angiogenic disorder in an individual in need thereof, comprising administering to the individual a VEGF variant polypeptide or an Fc-VEGF variant polypeptide fusion. In certain embodiments, disclosed herein, are kits comprising a VEGF variant polypeptide or Fc-VEGF variant polypeptides.

### CROSS-REFERENCE

This application is a 371 application and claims the benefit of PCT Application No. PCT/US2016/013688, filed Jan. 15, 2016, which claims the benefit of U.S. Provisional Patent Application Ser. No. 62/104,590, filed Jan. 16, 2015, and U.S. Provisional Patent Application Ser. No. 62/104,588, filed Jan. 16, 2015, and U.S. Provisional Patent Application Ser. No. 62/104,621, filed Jan. 16, 2015, each of which is incorporated by reference herein in its entirety.

### SUMMARY OF THE INVENTION

Disclosed herein, in certain embodiments, are VEGF variant polypeptides comprising a first VEGF monomer joined to a second VEGF monomer by a peptide linker or a disulfide bridge. In some embodiments, the VEGF variant polypeptides comprise the formula:

A-L-B,
wherein
   A is a first VEGF monomer subunit;
   B is a second VEGF monomer subunit; and
   L is a peptide linker having 14 to 20 amino acids.
      In some embodiments, L is a peptide linker having a formula selected from: (GS)ₙ, wherein n is an integer from 6 to 15; (G₂S)ₙ, wherein n is an integer from 4 to 10; (G₃S)ₙ, wherein n is an integer from 3 to 8; (G₄S)ₙ, wherein n is an integer from 2 to 6; (G)ₙ, wherein n is an integer from 12 to 30; and (S)ₙ, wherein n is an integer from 12 to 30. In some embodiments, L is selected from the group consisting of: GSTSGSGKSSEGKGGGGGS (SEQ ID NO: 42); GGGGSGGGGSGGGG (SEQ ID NO: 43); and GGGGSGGGGSGGGGSGGGGS (SEQ ID NO: 44). In some embodiments, the VEGF variant polypeptides comprise the formula:

A-L₁-B-(L₂-A-L₁-B)ₙ-L₂-A-L₁-B,
wherein
   A is a first VEGF monomer subunit,
   B is a second VEGF monomer subunit,
   L₁ is a peptide linker having 14 to 20 amino acids;
   L₂ is a peptide linker; and
   n is an integer from 0 to 4.
      In some embodiments, L₁ is a peptide linker having a formula selected from: (GS)ₙ, wherein n is an integer from 6 to 15; (G₂S)ₙ, wherein n is an integer from 4 to 10; (G₃S)ₙ, wherein n is an integer from 3 to 8; (G₄S)ₙ, wherein n is an integer from 2 to 6; (G)ₙ, wherein n is an integer from 12 to 30; and (S)ₙ, wherein n is an integer from 12 to 30. In some embodiments, L₁ is selected from the group consisting of: GSTSGSGKSSEGKGGGGGS (SEQ ID NO: 42); GGGGSGGGGSGGGG (SEQ ID NO: 43); and GGGGSGGGGSGGGGSGGGGS (SEQ ID NO: 44). In some embodiments, L₂ is selected from the group consisting of: (GS)ₙ, where n=10-30; (G₂S)ₙ, where n=6-20; (G₃S)ₙ, where n=5-15; (G₄S)ₙ, where n=4-12; (G)ₙ, where n=20-60; and (S)ₙ, where n=20-60.

In some embodiments, the VEGF variant polypeptide is a bifunctional antagonist. In some embodiments, the VEGF variant polypeptide antagonizes a VEGFR, an integrin, or combination thereof. In some embodiments, the VEGFR is VEGFR1. In some embodiments, the VEGFR is VEGFR2. In some embodiments, the integrin is αᵥβ₃, αᵥβ₅ or α₅β₁ integrin, or any combinations thereof. In some embodiments, at least one of the VEGF monomer subunits is a VEGF-A monomer. In some embodiments, the VEGF-A monomer is VEGF₁₆₅. In some embodiments, the VEGF-A monomer is VEGF_{166b}. In some embodiments, the VEGF-A monomer is VEGF₁₂₁. In some embodiments, the VEGF-A monomer is VEGF₁₄₅. In some embodiments, the VEGF-A monomer is VEGF₁₈₉. In some embodiments, the VEGF-A monomer is VEGF₂₀₆. In some embodiments, at least one of the VEGF monomer subunits is a VEGF-B subunit. In some embodiments, at least one of the VEGF monomer subunits is a VEGF-C subunit. In some embodiments, at least one of the VEGF monomer subunits is a VEGF-D subunit. In some embodiments, at least one of the VEGF monomer subunits is a PIGF. In some embodiments, the first VEGF monomer subunit and the second VEGF monomer subunit are each independently a VEGF-A monomer.

In some embodiments, the first VEGF monomer subunit comprises a mutation selected from the group consisting of: V14A, V14I, V15A, K16R, F17L, M18R, D19G, Q22R, R23K, I29V, L325, I35V, F36L, F36S, D41N, E42K, E44G, Y45H, F47S, K48E, P49L, S50P, P53S, G58S, C60Y, D63H, D63N, D63G, I76T, M78V, M81T, M81V, R82G, H86Y, Q87R, Q89H, H90R, I91T, I91V, N100D, and K101E. In some embodiments, the first VEGF monomer subunit comprises a mutation selected from the group consisting of F36L, E44G, D63G, and Q87R. In some embodiments, the first VEGF monomer subunit comprises a mutation selected from the group consisting of F36L, E44G, and Q87R. In some embodiments, the second VEGF monomer subunit comprises a mutation selected from the group consisting of V14A, V14I, V15A, K16R, F17L, M18R, D19G, Q22R, R23K, I29V, L32S, I35V, F36L, F36S, D41N, E42K, E44G, Y45H, F47S, K48E, P49L, S50P, P53S, G58S, C60Y, D63H, D63N, D63G, I76T, M78V, M81T, M81V, R82G, H86Y, Q87R, Q89H, H90R, I91T, I91V, N100D, and K101E. It will be understood by one of skill in the art that the designation throughout of “first” and “second” with respect to the VEGF monomers is an arbitrary distinction, and either chain can be “first” or “second”.

In some embodiments, the second VEGF monomer subunit comprises a mutation selected from the group consisting of K16R, D41N, and D63N. In some embodiments, the second VEGF monomer subunit comprises a mutation selected from the group consisting of D63N.

In some embodiments, the first or the second or both of the VEGF monomer subunits comprises an RGD loop. In some embodiments, the RGD loop is at least 90%, at least 95%, at least 99%, or 100% identical to a sequence selected from the group consisting of SEQ ID NOs: 1-40, 66-72. In some embodiments, the RGD containing loop replaces loop 1, loop 2, or loop 3 of the first or the second VEGF monomer subunit, or any combinations thereof.

In some embodiments, the VEGF variant polypeptide is at least 90%, at least 95%, at least 99%, or 100% identical to a sequence of mE7I (SEQ ID NO: 75). In some embodiments, the VEGF variant polypeptide is at least 90%, at least 95%, at least 99%, or 100% identical to a sequence of mA7I (SEQ ID NO: 76). In some embodiments, the VEGF variant polypeptide is at least 90%, at least 95%, at least 99%, or 100% identical to a sequence of mJ7I (SEQ ID NO: 77). In some embodiments, the VEGF variant polypeptide is at least 90%, at least 95%, at least 99%, or 100% identical to a sequence of mE7I-R1null (SEQ ID NO: 78).

In some embodiments, the VEGF variant polypeptide further comprises a toxin. In some embodiments, the toxin is selected from the group consisting of a Pseudomonas exotoxin (PE), a Diphtheria toxin (DT), ricin toxin, abrin toxin, anthrax toxins, shiga toxin, botulinum toxin, tetanus toxin, cholera toxin, maitotoxin, palytoxin, ciguatoxin, textilotoxin, batrachotoxin, alpha conotoxin, taipoxin, tetrodotoxin, alpha tityustoxin, saxitoxin, anatoxin, microcystin, aconitine, exfoliatin toxins A, exfoliatin B, an enterotoxin, toxic shock syndrome toxin (TSST-I), Y. pestis toxin and a gas gangrene toxin. In some embodiments, the toxin is attached to the N-terminus of the VEGF variant. In some embodiments, the toxin is attached to the C-terminus of the VEGF variant. In some embodiments, the toxin is attached to the first or the second VEGF monomer subunit.

Disclosed herein, in certain embodiments, are VEGF variant polypeptides of the formula A-L-B as defined above, comprising (a) a first VEGF-A monomer subunit having the following mutations: F36L, E44G, and Q87R or F36L, E44G, D63G, and Q87R (b) a second VEGF-A monomer subunit having the mutation: D63N, and (c) a peptide linker or a disulfide bridge joining the first and the second VEGF-A monomers.

Disclosed herein, in certain embodiments, are VEGF variant polypeptides comprising the formula:

A-L₁-B-(L₂-A-L₁-B)ₙ-L₂-A-L₁-B,

wherein A is a first VEGF-A monomer having the following mutations: F36L, E44G, and Q87R; B is a second VEGF-A monomer having the mutation D63N; L₁ is a peptide linker; L₂ is a peptide linker; and n is an integer from 0 to 4, and each of A and B are as defined above. In some embodiments, L₁ is 14 amino acids in length. In some embodiments, L₁ is 15 amino acids in length. In some embodiments, L₁ is 16 amino acids in length. In some embodiments, L₁ is 17 amino acids in length. In some embodiments, L₁ is 18 amino acids in length. In some embodiments, L₁ is 19 amino acids in length. In some embodiments, L₁ is 20 amino acids in length. In some embodiments, L₁ has at least 90%, 95%, 99% or 100% sequence identity to GSTSGSGKSSEGKG (SEQ ID NO: 41). In some embodiments, L₁ has at least 90%, 95%, 99% or 100% sequence identity to GGGGSGGGGSGGGGSGGGGS (SEQ ID NO: 44) In some embodiments, L₁ has at least 90%, 95%, 99% or 100% sequence identity to GSTSGSGKSSEGKGGGGGS (SEQ ID NO:42). In some embodiments, L₁ has at least 90%, 95%, 99% or 100% sequence identity to GGGGSGGGGSGGGG (SEQ ID NO:43). In some embodiments, L₂ is selected from the group consisting of: (GS)ₙ, where n=10-30; (G₂S)ₙ, where n=6-20; (G₃S)ₙ, where n=5-15; (G₄S)ₙ, where n=4-12; (G)ₙ, where n=20-60; and (S)ₙ where n=20-60. In some embodiments, the VEGF variant polypeptide is at least 90%, at least 95%, at least 99%, or 100% identical to a sequence of mE7l (SEQ ID NO: 75).

In certain embodiments an VEGF variant polypeptide as defined above is fused to an immunoglobulin Fc region to generate an Fc-VEGF variant polypeptide. The Fc-VEGF variant polypeptide fusion may comprise the formula:

Fc-(A-L-B), or (A-L-B)-Fc

wherein
Fc is an immunoglobulin Fc region;
A and B are each independently a VEGF monomer; and
L is a peptide linker amino acids, each of A, B and L as defined above.

Disclosed herein, in certain embodiments, are Fc-VEGF variant polypeptide fusions comprising the formula:

Fc-[A-L₁-B-(L₂-A-L₁-B)ₙ-L₂-A-L₁-B], or Fc-[A-L₁-B-(L₂-A-L₁-B)ₙ-L₂-A-L₁-B]

wherein
Fc is an immunoglobulin Fc region;
A is a first VEGF monomer;
B is a second VEGF monomer; and
L₁ and L₂ are each independently a peptide linker, each of A, B L₁ and L₂ as defined above; and
n is an integer from 0 to 4.

Compositions include one or more variant VEGF polypeptide(s) of the invention, which may be provided as a single species or as a cocktail of two or more polypeptides, usually in combination with a pharmaceutically acceptable excipient. Such compositions optionally comprise one or more additional therapeutic agents. Pharmacologic compositions comprise one or more polypeptides of the invention and a pharmaceutically acceptable excipient. Compositions can be provided for topical or systemic use. In some embodiments, the pharmaceutical composition is a topical composition. In some embodiments, the pharmaceutical composition is a locally injected composition into the skin, ocular tissue, cerebrospinal fluid, tumor, joint space, etc. In some embodiments, the pharmaceutical composition is a systemic composition delivered orally or intravenously. In some embodiments, the pharmaceutical composition is an eye drop. In some embodiments, the pharmaceutical composition is formulated as an ophthalmically acceptable solution, cream or ointment. Ophthalmic compositions of the invention can be formulated for non-surgically treating a disorder characterized by neovascularization of the external surface of the eye, including the cornea and bulbar conjunctiva, in a subject in need thereof. In some embodiments the composition is formulated for preventing recurrence of a disorder characterized by neovascularization of the external surface of the eye, including the cornea and bulbar conjunctiva, in a subject in need thereof. In some embodiments, the composition is formulated for intraocular injection, subconjunctival injection, or periocular injection.

In some embodiments the polypeptide of the invention is conjugated to a functional moiety, e.g. a detectable label such a fluorescent label, a detectable label such as an isotopic label; a cytotoxic moiety, and the like, which may find use in imaging, quantitation, therapeutic purposes, etc. In some embodiments, the hybrid polypeptide of the present invention further comprises a toxin. In some embodiments, the toxin is selected from the group consisting of a Pseudomonas exotoxin (PE), a Diphtheria toxin (DT), ricin toxin, abrin toxin, anthrax toxins, shiga toxin, botulism toxin, tetanus toxin, cholera toxin, maitotoxin, palytoxin, ciguatoxin, textilotoxin, batrachotoxin, alpha conotoxin, taipoxin, tetrodotoxin, alpha tityustoxin, saxitoxin, anatoxin, microcystin, aconitine, exfoliatin toxins A, exfoliatin B, an enterotoxin, toxic shock syndrome toxin (TSST-I), Y. pestis toxin and a gas gangrene toxin. In some embodiments, the toxin is attached to the N-terminus of the polypeptide. In some embodiments, the toxin is attached to the C-terminus of the polypeptide. In some embodiments, the toxin is attached to the PDGF chain, the VEGF chain, or both.

Disclosed herein, in certain embodiments, are methods of treating an angiogenic disorder in an individual in need thereof, comprising administering to the individual a VEGF variant polypeptide disclosed herein or an Fc-VEGF variant polypeptide fusion disclosed herein. In some embodiments, the angiogenic disorder is pterygium. In some embodiments, the angiogenic disorder is corneal neovascularization. In some embodiments, the angiogenic disorder is macular degeneration. In some embodiments, the angiogenic disorder is retinal vein occlusion. In some embodiments, the angiogenic disorder is ocular neovascularization, choroidal neovascularization, iris neovascularization, corneal neovascularization, retinal neovascularization, pinguecula, pannus, diabetic retinopathy (DR), diabetic macular edema (DME), retinal detachment, posterior uveitis, diabetic retinopathy, macular degeneration, for example, age-related macular degeneration (AMD), particularly wet macular degeneration, keloid, glaucoma, cataract, partial blindness, complete blindness, myopia, myopic degeneration, deterioration of central vision, metamophopsia, color disturbances, hemorrhaging of blood vessels, or a combination thereof. In some embodiments, the subject has a fibrovascular growth, including but not limited to pterygium.

In some embodiments, the angiogenic disorder is a cancer. In some embodiments, the cancer is prostate cancer, breast cancer, lung cancer, esophageal cancer, colon cancer, rectal cancer, liver cancer, urinary tract cancer (e.g., bladder cancer), kidney cancer, lung cancer (e.g., non-small cell lung cancer), ovarian cancer, cervical cancer, endometrial cancer, pancreatic cancer, stomach cancer, thyroid cancer, skin cancer (e.g., melanoma), hematopoietic cancers of lymphoid or myeloid lineage, head and neck cancer, nasopharyngeal carcinoma (NPC), glioblastoma, teratocarcinoma, neuroblastoma, adenocarcinoma, cancers of mesenchymal origin such as a fibrosarcoma or rhabdomyosarcoma, soft tissue sarcoma and carcinoma, choriocarcinioma, hepatoblastoma, Karposi's sarcoma or Wilm's tumor. In some embodiments, the angiogenic disorder is an inflammatory disorder. In some embodiments, the inflammatory disorder is inflammatory arthritis, osteoarthritis, psoriasis, chronic inflammation, irritable bowel disease, lung inflammation or asthma. In some embodiments, the angiogenic disorder is an autoimmune disorder. In some embodiments, the autoimmune disease is rheumatoid arthritis, multiple sclerosis, or systemic lupus erythematosus. In some embodiments, the angiogenic disorder is atherosclerosis, retrolentral fibroplasia, thyroid hyperplasias (including grave's disease), nephrotic syndrome, preclampasia, ascites, pericardial effusion (such as associated with pericarditis) and pleural effusion.

In some embodiments, methods are provided for non-surgically treating a disorder characterized by neovascularization of the external surface of an eye, including the cornea and bulbar conjunctiva, of a subject in need thereof, comprising administering to the subject an effective amount of a pharmaceutical composition comprising a hybrid polypeptide of the present invention. In some embodiments, methods are provided for preventing recurrence of neovascularization of the external surface of an eye, including the cornea and bulbar conjunctiva, of a subject in need thereof, comprising administering to the subject an effective amount of a pharmaceutical composition comprising a hybrid polypeptide of the present invention.

In some embodiments, the method comprises administering an additional therapeutic agent. In some embodiments, the additional therapeutic agent is selected from the group consisting of an antibody, polypeptide, nucleotide, a small molecule, and combinations thereof. In some embodiments, the additional therapeutic agent is an inhibitor of a VEGF, an inhibitor of a PDGF, an inhibitor of an ANG, or an inhibitor of a FGF, or associated receptors. In some embodiments, the additional therapeutic agent is an inhibitor of an integrin, or an inhibitor of a MMP, or an inhibitor of prostate specific membrane antigen (PSMA). In some embodiments, the additional therapeutic agent is selected from the group consisting of: mitomycin C (MMC), 5-fluorouracil (5-FU), loteprednol etabonate (LE), oral doxycycline, dipyridamole, and dobesilate. In some embodiments, the additional therapeutic agent is an anti-inflammatory steroid. In some embodiments, the additional therapeutic agent is non-steroidal anti-inflammatory agent. In some embodiments, the additional therapeutic agent is an antibody or small molecule inhibitor of VEGF signaling. In some embodiments, the additional therapeutic agent binds, traps, scavenges or otherwise deters the effect of VEGF that has already been produced. The additional therapeutic agent can be formulated in the pharmaceutical composition, including ophthalmic compositions, with the hybrid polypeptide of the invention, or can be administered in a separate formulation.

In some embodiments, the disorder characterized by neovascularization of the external surface of the eye is pterygium. In some embodiments, the pterygium is chronic pterygium. In some embodiments, the pterygium is recurrent pterygium. In some embodiments, the disorder characterized by neovascularization of the external surface of the eye is pannus. In some embodiments, the disorder characterized by neovascularization of the external surface of the eye is corneal neovascularization. In some embodiments, the disorder characterized by neovascularization of the external surface of the eye is pinguecula. In some embodiments, the disorder characterized by neovascularization at the limbus of the cornea caused by contact lens overwear. In some embodiments, the disorder has not healed within one month of a surgical intervention. In some embodiments, the hybrid polypeptide of the present invention is administered during or after a surgical intervention or debridement.

### BRIEF DESCRIPTION OF THE DRAWING

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 shows an image of a gel showing the protein yield for constructs with different peptide linkers (L1A, L2A, and L3A).

FIG. 2 shows a plot of the results of a cell binding assay on human endothelial cells performed to compare target binding affinity of a construct containing peptide linker L3A compared to the original linker.

FIG. 3 shows a plot of VEGFR binding versus expression for a library of VEGF variant polypeptides derived from scVEGF_{MUT}-E.

FIG. 4 shows a plot of binding of Fc-fusions of scVEGF constructs.

FIG. 5 shows a plot of the results of a phosphorylation assay on HUVECs.

FIG. 6 exemplifies a single-chain VEGF variant polypeptide blocking angiogenesis in an experimental model of corneal neovascularization.

FIG. 7 exemplifies immunohistochemical staining of von Willebrand Factor (vWF) and VEGFR2 in human pterygium.

FIG. 8 exemplifies immunohistochemical staining of vWF and VEGFR1 in human pterygium.

FIG. 9 exemplifies immunohistochemical staining of αᵥβ₃ integrin and VEGFR2 in human pterygium.

FIG. 10 exemplifies immunohistochemical staining of CD31 and α₅β₁ integrin in human pterygium.

FIG. 11 exemplifies immunohistochemical staining of CD31, and αᵥβ₅ integrin in human pterygium.

FIG. 12 exemplifies immunohistochemical staining of MMP2, pro-MMP2, and CD31 in human pterygium.

### DETAILED DESCRIPTION

Several embodiments are described below with reference to example applications for illustration. It should be understood that numerous specific details, relationships, and methods are set forth to provide a full understanding of the features described herein. A skilled artisan in the relevant art, however, will readily recognize that the features described herein, in some embodiments, are practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

“Amino acid” refers to naturally occurring amino acids, non-naturally occurring amino acids, and amino acid analogs, and to the D or L stereoisomers of each.

The terms “peptide”, “polypeptide”, and “amino acid sequence” refer to a chain of amino acids. “Peptide”, “polypeptide”, and “amino acid sequence” are used interchangeably.

The terms “peptide linker”, “polypeptide linker” or “amino acid” refer to a chain of amino acids that link one VEGF monomer subunit to another VEGF monomer subunit. The terms are used interchangeably.

VEGF, or Vascular Endothelial Growth Factor, refers to a family of signaling proteins that stimulate angiogenesis, vasculogenesis and lymphangiogenesis. Members of the VEGF family include VEGF-A, VEGF-B, VEGF-C, VEGF-D, and PIGF (Placental Growth Factor). If the particular member of VEGF is not specified, then VEGF means any of VEGF-A, VEGF-B, VEGF-C, VEGF-D, and PIGF. Within this application, amino acid residue numbering of any VEGF monomer commences at residue 13 with respect to the mature human wild type VEGF-A sequence. SEQ ID No.: 73 is the mature full length sequence of VEGF 121. SEQ ID No.: 74 is a fragment of the mature full length VEGF 121 that contains both an N-terminal truncation of the first 12 amino acid residues (consequently numbering begins at 13), and a C-terminal truncation of the last 12 amino acid residues. When referred to herein, Loop 1 of VEGF-A means amino acid residues 62 to 67 (with respect to the mature human wild type VEGF-A sequence); Loop 2 means amino acid residues 39 to 46 (with respect to the mature human wild type VEGF-A sequence); and Loop 3 means amino acid residues 83-89 (with respect to the mature human wild type VEGF-A sequence). Loops 1, 2, and 3 of other VEGF family members can be similarly defined or inferred by homology.

A “VEGF monomer subunit” means a VEGF monomer amino acid sequence. In some embodiments, a VEGF monomer subunit has the sequence of SEQ ID No.: 73. In some embodiments, a VEGF monomer subunit has the sequence of SEQ ID No.: 74. In some embodiments, a VEGF monomer subunit has the sequence of SEQ ID No.: 73, wherein the sequence of SEQ ID No. 73 is modified with one or more mutations (e.g., a replacement, addition, insertion, omission, substitution or deletion, or a combination thereof). In some embodiments, a VEGF monomer subunit has the sequence of SEQ ID No.: 74, wherein the sequence of SEQ ID No. 74 is modified with mutations (e.g., a replacement, addition, insertion, omission, substitution or deletion, or a combination thereof). In some embodiments, a VEGF monomer subunit has the sequence of SEQ ID No.: 73, wherein loop 1, loop 2 or loop 3 of SEQ ID No.: 73, or any combinations thereof, has been replaced with a heterologous motif (e.g., an RGD recognition motif). In some embodiments, a VEGF monomer subunit has the sequence of SEQ ID No.: 74, wherein loop 1, loop 2 or loop 3 of SEQ ID No.: 74, or any combinations thereof, has been replaced with a heterologous motif (e.g., an RGD recognition motif).

A “VEGF variant polypeptide” refers to a molecule comprising at least two VEGF monomer subunits associated together, for example by a linker or a disulfide bridge. In some embodiments, one or both linked VEGF monomer subunits contain one or more mutations.

“scVEGF variant” describes a single-chain version of a VEGF variant polypeptide, i.e. a single chain molecule in which two VEGF monomer subunits are joined for example by a peptide linker. As used herein, the terms “single chain VEGF variant”, and “scVEGF variant” are used interchangeably.

As used herein, “pole” or “face” refers to a VEGFR binding interface of a VEGF variant polypeptide. The “pole” or “face” comprises amino acids residues from the first VEGF monomer subunit and the second VEGF monomer subunit. Each pole binds to one VEGFR molecule. “Pole” and “face” are used interchangeably.

“Mutant” refers to a polypeptide that differs in some way from a reference wild-type polypeptide. The polypeptide retains biological properties of the reference wild-type (e.g., naturally occurring) polypeptide. In some embodiments, the polypeptide has biological properties that differ from the reference wild-type polypeptide. In some embodiments, the mutant has a mutation in which the polypeptide chain has a replacement, addition, insertion, omission, substitution or deletion, or a combination thereof of the amino acid residues.

An “anti-VEGF agent” means an inhibitor of VEGF signaling, for example a competitive antagonist, a non-competitive antagonist, an uncompetitive antagonist, a silent antagonist, a partial agonist, or an inverse agonist.

“Purified” or “substantially purified” denotes that the indicated molecule is present in the substantial absence of other biological macromolecules, for example, polynucleotides, proteins, and the like. In some embodiments, the molecule is purified such that it constitutes at least 95% by weight, for example, at least 99% by weight, of the indicated biological macromolecules present. In some embodiments, water, buffers, and other small molecules with a molecular weight of less than 1000 Daltons, are present in any amount.

“Isolated” as used herein refers to a molecule separated from at least one other component present with the molecule in its natural source. In some embodiments, the molecule is isolated such that it constitutes greater than 50% by weight, for example, at least 75% by weight, of the indicated biological macromolecules present.

The terms “individual,” “patient,” or “subject” are used interchangeably. As used herein, they mean any mammal (i.e. species of any orders, families, and genus within the taxonomic classification animalia: chordata: vertebrate: mammalia). In some embodiments, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

“Treating” or “treatment” of a state, disorder or condition (e.g., pterygium) includes: (1) preventing or delaying the appearance of clinical or sub-clinical symptoms of the state, disorder or condition developing in a mammal that is afflicted with or predisposed to the state, disorder or condition but does not yet experience or display clinical or subclinical symptoms of the state, disorder or condition; and/or (2) inhibiting the state, disorder or condition, including arresting, reducing or delaying the development of the disease or a relapse thereof (in case of maintenance treatment) or at least one clinical or sub-clinical symptom thereof; and/or (3) relieving the disease, e.g., causing regression of the state, disorder or condition or at least one of its clinical or sub-clinical symptoms; and/or (4) causing a decrease in the severity of one or more symptoms of the disease. The benefit to a subject to be treated is either statistically significant or at least perceptible to the patient or to the physician.

“Angiogenic disorder” as used herein, means any condition or disorder that is associated with or that results from pathological angiogenesis, or that is facilitated by neovascularization (e.g., a tumor that is dependent upon neovascularization).

### VEGF Variant Polypeptides

Disclosed herein, in some embodiments, are VEGF variant polypeptides. In some embodiments, the VEGF variant polypeptides are Fc fusions. In some embodiments, such VEGF variant polypeptides are used in methods of diagnosing and treating an angiogenic disorder, for example, an angiogenesis associated eye disorder. In some embodiments, the VEGF variant polypeptides are used in treating pterygium.

A VEGF variant polypeptide, as disclosed herein, is a molecule comprising at least two VEGF monomer subunits joined together, for example by a linker. In some embodiments, one or both linked VEGF monomer subunits contain one or more mutations, for example a replacement, addition, insertion, omission, substitution or deletion, or a combination thereof of the amino acid residues.

In some embodiments, the VEGF variant polypeptide is a VEGF receptor antagonist. In some embodiments, a VEGF variant polypeptide is an integrin receptor antagonist. In some embodiments, a VEGF variant polypeptide is an integrin receptor antagonist and VEGF receptor antagonist. In some embodiments, the VEGF variant polypeptide is a vitronectin receptor antagonist. In some embodiments, the VEGF variant polypeptide is a vitronectin receptor antagonist and a VEGF receptor antagonist.

In some embodiments, one pole of the VEGF variant polypeptide comprises an intact VEGFR binding site such that this pole is able to bind to VEGFR. In some embodiments, at least one pole of the VEGF variant polypeptide is not able to bind to a VEGFR. In some embodiments, upon binding of the VEGF variant polypeptide to a VEGFR, the VEGFR is not activated. This thereby antagonizes VEGF-stimulated receptor autophosphorylation and propagation of downstream signaling resulting in inhibition of angiogenesis. Without being bound to any one theory, the VEGF variant polypeptides disclosed herein are able to antagonize a VEGFR and subsequent signaling induced by VEGFR activation, because one pole of the VEGF variant polypeptide has an intact VEGFR binding site. This pole of the VEGF variant polypeptide is able to bind to a VEGFR, while the other pole of the VEGF variant polypeptide contains at least one mutation such that it cannot bind to a second VEGFR, which prevents VEGFR dimerization and activation.

In some embodiments, at least one of the VEGF monomer subunits is VEGF-A. In some embodiments, at least one of the VEGF monomer subunits is the VEGF-A isoform. In some embodiments, the VEGF-A isoform is 121, 145, 148, 165, 183, 189, or 206 amino acids. In some embodiments, the VEGF-A isoform is the VEGF₁₆₅b isoform. In some embodiments, at least one of the VEGF monomer subunits is VEGF-B, VEGF-C, VEGF-D or PIGF. Any suitable VEGF monomer subunit is contemplated for use with the methods disclosed herein. In some embodiments, the VEGF variant polypeptide is derived from the monomer VEGF-A₁₂₁, but contains only the 97-amino acid core region of VEGF-A₁₂₁ (see SEQ ID NO: 74).

In some embodiments, VEGF variant polypeptides have a truncated N-terminus, C-terminus, or both, relative to a VEGF monomer subunit.

### VEGF Variant Fusion Polyeptides

In some embodiments, a VEGF variant polypeptide further comprises at least one other molecule, including, but not limited to other cysteine knot growth factors or glycoproteins. For instance, in some embodiments, the fusion peptide comprises a VEGF-A monomer fused to a VEGF-B, VEGF-C, VEGF-D, VEGF-E, VEGF-F, PDGF or PIGF monomer; a VEGF-B monomer is fused to a VEGF-A, VEGF-C, VEGF-D, VEGF-E, VEGF-F, PDGF or PIGF monomer; a VEGF-C monomer is fused to a VEGF-A, VEGF-B, VEGF-D, VEGF-E, VEGF-F, PDGF or PIGF monomer; a VEGF-D monomer is fused to a VEGF-A, VEGF-B, VEGF-C, VEGF-E, VEGF-F, PDGF or PIGF monomer; or a PIGF monomer is fused to a VEGF-A, VEGF-B, VEGF-C, VEGF-D, VEGF-E, VEGF-F, or PDGF monomer.

In some embodiments, the VEGF variant polypeptide is attached to a toxin, for example by a covalent or ionic bond. In some embodiments, a VEGF variant polypeptide is attached to a toxin by a peptide bond. In some embodiments, the toxin is attached to the N-terminus of the VEGF variant polypeptide. In some embodiments, the toxin is attached to the C-terminus of the VEGF variant polypeptide. In some embodiments, the toxin is attached to the first or the second VEGF monomer subunit.

In some embodiments, the toxin is selected from the group consisting of: pseudomonas exotoxin (PE), a Diphtheria toxin (DT), ricin toxin, abrin toxin, anthrax toxins, shiga toxin, botulism toxin, tetanus toxin, cholera toxin, maitotoxin, palytoxin, ciguatoxin, textilotoxin, batrachotoxin, alpha conotoxin, taipoxin, tetrodotoxin, alpha tityustoxin, saxitoxin, anatoxin, microcystin, aconitine, exfoliatin toxins A, exfoliatin B, an enterotoxin, toxic shock syndrome toxin (TSST-I), Y. pestis toxin and a gas gangrene toxin.

In some embodiments, a VEGF variant polypeptide comprises an Fc-fusion. In some embodiments, the C-terminus of scVEGF is joined to N-terminus of Fc. In some embodiments, the C-terminus of Fc is fused to N-terminus of scVEGF. In some embodiments, the Fc-fusion is naturally occurring or engineered. In some embodiments, the Fc-fusion is from human, mouse, rat, and rabbit. In some embodiments, the VEGF variant polypeptide comprising an Fc-fusion induces involvement of immune cells. In some embodiments, the VEGF variant polypeptide comprising an Fc-fusion binds to Fc receptors. In some embodiments, the VEGF variant polypeptide comprising an Fc-fusion induces involvement of an immune cell. In some embodiments, the immune cell is B lymphocytes, follicular dendritic cells, natural killer cells, macrophages, neutrophils, eosinophils, basophils, and mast cells. In some embodiments, the VEGF variant polypeptide comprising an Fc-fusion does not have altered binding affinity to VEGFR or integrin from the VEGF variant polypeptide without the Fc-fusion. In some embodiments, the VEGF variant polypeptide comprising an Fc-fusion does not have altered antagonistic activity to VEGFR or integrin from the VEGF variant polypeptide without the Fc fusion. In some embodiments, the VEGF variant polypeptide comprising an Fc-fusion has enhanced binding affinity to VEGFR or integrin from the VEGF variant polypeptide without the Fc-fusion. In some embodiments, the VEGF variant polypeptide comprising an Fc-fusion has enhanced antagonistic activity to VEGFR or integrin from the VEGF variant polypeptide without the Fc fusion. In some embodiments, the VEGF variant polypeptide is connected to the Fc-fusion by a Gly₄Ser linker at the fusion junction of the VEGF variant polypeptide and the Fc-fusion. In some embodiments, the VEGF variant polypeptide is connected to the Fc-fusion without a Gly₄Ser linker. In some embodiments, the Gly₄Ser linker comprises one Gly₄Ser repeat. In some embodiments, the Gly₄Ser linker comprises two Gly₄Ser repeats. In some embodiments, the Gly₄Ser linker comprises three Gly₄Ser repeats.

### VEGF Variant Polypeptides with Heterologous Motifs

In some embodiments, a VEGF variant polypeptide comprises a heterologous motif that binds to a non-VEGFR protein. In some embodiments, the first or the second VEGF peptide monomer subunit comprises a heterologous motif that binds to a non-VEGFR protein. In some embodiments, the first and the second VEGF peptide monomer subunits each independently comprise a heterologous motif that binds to a non-VEGFR protein. In some embodiments, a single heterologous motif is divided between the first and the second VEGF peptide monomer subunits. In some embodiments, the non-VEGFR protein is a receptor. In some embodiments, the non-VEGFR protein is a vascular protein. In some embodiments, the VEGF variant polypeptide comprising a heterologous motif has an increased affinity for a VEGFR2 relative to the wild-type VEGF.

In some embodiments, the non-VEGF protein is an integrin. Integrins are a diverse class of heterodimeric (α/β) receptors involved in cell adhesion to extracellular matrix ligands. In particular, integrin αᵥβ₃ has been implicated as critically involved in tumor proliferation, metastasis, and angiogenesis, and there have therefore been many efforts to develop anti-cancer therapies that target integrin αᵥβ₃. Human pterygium tissue samples are positive for αᵥβ₃, αᵥβ₅ and α₅β₁.

In some embodiments, a VEGF variant polypeptide is a bispecific protein targeting both VEGFR2 and αᵥβ₃ integrin. In some embodiments, a VEGF variant polypeptide is a multispecific antagonist targeting VEGFR1, VEGFR2 and αᵥβ₃ integrin. In some embodiments, a VEGF variant polypeptide comprises a loop carrying an integrin-recognition RGD sequence for binding of αᵥβ₃ integrin in the mutated receptor binding site, thereby antagonizing not only VEGF-stimulated proliferation of endothelial cells, but also activation of αᵥβ₃ integrin.

In some embodiments, a VEGF variant polypeptide comprises one intact and one mutated VEGF receptor binding pole, wherein the mutated binding pole contains a loop with an integrin-recognition RGD sequence for binding of an integrin, for example αᵥβ₃, αᵥβ₅ or α₅β₁ integrin. In some embodiments, the integrin-recognition RGD sequence replaces loop 1, loop 2, or loop 3 of the VEGF monomer subunit. In some embodiments the loop 1 sequence is replaced with the RGD motif. In some embodiments the loop 2 sequence is replaced with the RGD motif. In some embodiments the loop 3 sequence is replaced with the RGD motif. In some embodiments the loop 3 sequence (SEQ ID NO: 64) IKPHQGQ is replaced with the RGD motif. Table 1 shows sequences of exemplary integrin-binding loop peptides.

TABLE 1
Exemplary integrin-binding loop peptides.

| SEQ ID NO: | Grafted Loop Sequence |
|---|---|
| SEQ ID NO: 1 | PFGTRGDSS |
| SEQ ID NO: 2 | SGERGDGPT |
| SEQ ID NO: 3 | SDGRGDGSV |
| SEQ ID NO: 4 | PIGRGDGST |
| SEQ ID NO: 5 | LAERGDSSS |
| SEQ ID NO: 6 | PTGRGDLGA |
| SEQ ID NO: 7 | RGIRGDSGA |
| SEQ ID NO: 8 | VGGRGDVGV |
| SEQ ID NO: 9 | ITARGDSFG |
| SEQ ID NO: 10 | ITERGDSGH |
| SEQ ID NO: 11 | PQARGDRSD |
| SEQ ID NO: 12 | SRTRGDASD |
| SEQ ID NO: 13 | PAARGDGGL |
| SEQ ID NO: 14 | PVARGDSGA |
| SEQ ID NO: 15 | PQQRGDGPH |
| SEQ ID NO: 16 | PLPRGDGQR |
| SEQ ID NO: 17 | HAGRGDSPS |
| SEQ ID NO: 18 | TSLRGDTTW |
| SEQ ID NO: 19 | PNFRGDEAY |
| SEQ ID NO: 20 | AGVPRGDSP |
| SEQ ID NO: 21 | PRSTRGDST |
| SEQ ID NO: 22 | PFGVRGDDN |
| SEQ ID NO: 23 | GFPFRGDSPAS |
| SEQ ID NO: 24 | PSVRRGDSPAS |
| SEQ ID NO: 25 | PFAVRGDRP |
| SEQ ID NO: 26 | PWPRRGDLP |
| SEQ ID NO: 27 | PSGGRGDSP |
| SEQ ID NO: 28 | VGGRGDVGV |
| SEQ ID NO: 29 | ITSRGDHGE |
| SEQ ID NO: 30 | PPGRGDNGG |
| SEQ ID NO: 31 | PVARGDSGA |
| SEQ ID NO: 32 | STDRGDASA |
| SEQ ID NO: 33 | LNPRGDANT |
| SEQ ID NO: 34 | PSVRRGDSPAS |
| SEQ ID NO: 35 | PTTRGDCPD |
| SEQ ID NO: 36 | PGGRGDSAY |
| SEQ ID NO: 37 | PHDRGDAGV |
| SEQ ID NO: 38 | STDRGDASA |
| SEQ ID NO: 39 | ASGRGDGGV |
| SEQ ID NO: 40 | PASRGDSPP |

In addition, in some embodiments, a VEGF variant polypeptide comprises two or more RGD-containing loops, to enable binding to and inhibition of two or more specific integrins.

In some embodiments, a VEGF variant polypeptide comprises a heterologous motif that binds to a non-VEGFR protein. In some embodiments, the VEGF variant polypeptide comprises a heterologous motif that binds to a vascular protein. In some embodiments, the vascular protein is selected from the group consisting of: prostate-specific membrane antigen (PSMA), matrix metalloprotineases (MMPs), platetlet-derived growth factor receptor (PDGFR), platetlet-derived growth factor (PDGF), fibroblast growth factor receptor (FGFR), fibroblast growth factor (FGF) and the like. In some embodiments, the VEGF variant polypeptide comprises the cyclic decapeptide CTTHWGFTLC (SEQ ID NO: 65) which (i) inhibits the activities of MMP-2 and MMP-9, (ii) suppresses migration of both tumor cells and endothelial cells in vitro, (iii) home to tumor vasculature in vivo, and (iv) prevents the growth and invasion of tumors in mice. SEQ ID NO: 65 CTTHWGFTLC-displaying phage was also able to specifically target angiogenic blood vessels in vivo.

### Amino Acid Substitutions

In some embodiments, the first VEGF monomer subunit of the VEGF variant polypeptide comprises one or more mutations. In some embodiments, the second VEGF monomer subunit of the VEGF variant polypeptide comprises one or more mutations. In some embodiments, the first and second VEGF monomer subunits of the VEGF variant polypeptide each independently comprise one or more mutations.

In some embodiments, the VEGF variant polypeptide comprises at least one amino acid substitution in at least one VEGF monomer subunit. In some embodiments, the VEGF variant polypeptide comprises at least two amino acid substitutions, at least 3 amino acid substitutions, at least 4 amino acid substitutions or at least 5 amino acid substitutions in at least one or both of the VEGF monomer subunits. In addition to naturally occurring amino acids, non-naturally occurring amino acids, or modified amino acids, are also contemplated and within the scope.

In some embodiments, the substitutions are conservative amino acid substitutions, in which the substituted amino acid has similar structural or chemical properties with the corresponding amino acid in the reference sequence. In some embodiments, substitutions are non-conservative. For example, conservative amino acid substitutions involve substitution of one aliphatic or hydrophobic amino acids, e.g., alanine, valine, leucine and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine and histidine, with another; and replacement of one small amino acid, e.g., alanine, serine, threonine, methionine, and glycine, with another.

In some embodiments, the VEGF variant polypeptide comprises a portion of a full length active monomer, e.g., peptides that are not full length proteins. In some embodiments, the portion of a full length active monomer is obtained by substitution, replacement, addition, insertion, omission and/or deletion of an amino acid of these amino acid sequences. In some embodiments, the portion of a full length active monomer is linked with other peptides or polypeptides or with further chemical groups such as glycosyl groups, lipids, phosphates, acetyl groups or the like.

In some embodiments, one or both of the VEGF monomer subunits are mammalian VEGF peptides. In some embodiments, one or both of the VEGF monomer subunits are avian VEGF peptides. In some embodiments, one or both of the VEGF monomer subunits are primate VEGF peptides. In some embodiments, one or both of the VEGF monomer subunits are canine VEGF peptides. In some embodiments, one or both of the VEGF monomer subunits are feline VEGF peptides. In some embodiments, one or both of the VEGF monomer subunits are bovine VEGF peptides. In some embodiments, one or both of the VEGF monomer subunits are equine VEGF peptides. In some embodiments, one or both of the VEGF monomer subunits are porcine VEGF peptides. In some embodiments, one or both of the VEGF monomer subunits are ovine VEGF peptides. In some embodiments, one or both of the VEGF monomer subunits are murine VEGF peptides. In some embodiments, one or both of the VEGF monomer subunits are rat VEGF peptides. In some embodiments, one or both of the VEGF monomer subunits are rabbit VEGF peptides. In some embodiments, one or both of the VEGF monomer subunits are human VEGF peptides.

In some embodiments, a VEGF variant polypeptide comprises a first VEGF-A monomer and a second VEGF-A monomer. In some embodiments, the first VEGF-A monomer comprises a mutation selected from the group consisting of: V14A, V14I, V15A, K16R, F17L, M18R, D19G, Q22R, R23K, I29V, L32S, I35V, F36L, F36S, D41N, E42K, E44G, Y45H, F47S, K48E, P49L, S50P, P53S, G58S, C60Y, D63H, D63N, D63G, I76T, M78V, M81T, M81V, R82G, H86Y, Q87R, Q89H, H90R, I91T, I91V, N100D, and K101E. In some embodiments, the first VEGF-A monomer comprises a mutation selected from the group consisting of F36L, E44G, D63G, and Q87R. In some embodiments, the first VEGF-A monomer comprises the mutations of F36L, E44G, and Q87R. In some embodiments, the second VEGF-A monomer comprises a mutation selected from the group consisting of V14A, V14I, V15A, K16R, F17L, M18R, D19G, Q22R, R23K, I29V, L32S, I35V, F36L, F36S, D41N, E42K, E44G, Y45H, F47S, K48E, P49L, S50P, P53S, G58S, C60Y, D63H, D63N, D63G, I76T, M78V, M81T, M81V, R82G, H86Y, Q87R, Q89H, H90R, I91T, I91V, N100D, and K101E. In some embodiments, the second VEGF-A monomer comprises a mutation selected from the group consisting of K16R, D41N, and D63N. In some embodiments, the second VEGF-A monomer comprises the mutation D63N.

### Peptide Linkers

In some embodiments, a VEGF variant polypeptide comprises two or more VEGF monomer subunits separated by a peptide linker. A peptide linker is used to form a VEGF variant polypeptide in a single chain conformation. In some embodiments, a peptide linker does not hinder the ability of the single chain molecule to bind a VEGF receptor. In some embodiments, a peptide linker does not hinder the ability of the single chain molecule to bind an integrin receptor.

In some embodiments, the peptide linker ranges from about 2 to about 50 or more amino acids in length. For instance, in some embodiments, the peptide linker comprises about 2, 3, 4, 5, 6, 7, 8, 9, 10, 10-15, or 15-20 amino acids. In some embodiments, the peptide linker is 14-20 amino acids. In some embodiments, the peptide linker is 14 amino acids. In some embodiments, the peptide linker is 15 amino acids. In some embodiments, the peptide linker is 16 amino acids. In some embodiments, the peptide linker is 17 amino acids. In some embodiments, the peptide linker is 18 amino acids. In some embodiments, the peptide linker is 19 amino acids. In some embodiments, the peptide linker is 20 amino acids.

In some embodiments, the peptide linker is Gly-Ser or contains Gly-Ser. In some embodiments, the peptide linker is a glycine-rich polypeptide chain.

In some embodiments, the peptide linker sequence is GSTSGSGKSSEGKG (SEQ ID NO: 41). In some embodiments, the peptide linker sequence is GSTSGSGKSSEGKGGGGGS (SEQ ID NO: 42). In some embodiments, the peptide linker sequence is GGGGSGGGGSGGGG (SEQ ID NO: 43). In some embodiments, the peptide linker sequence is GGGGSGGGGSGGGGSGGGGS (SEQ ID NO: 44).

In some embodiments, the peptide linker comprises a peptide having the formula selected from the group: (GS)ₙ, wherein n is an integer from 6 to 15; (G₂S)ₙ, wherein n is an integer from 4 to 10; (G₃S)ₙ, wherein n is an integer from 3 to 8; (G₄S)ₙ, wherein n is an integer from 2 to 6; (G)ₙ, wherein n is an integer from 12 to 30; and (S)ₙ, wherein n is an integer from 12 to 30.

In some embodiments, the peptide linker is (Gly₄-Ser)₃ (SEQ ID NO: 45). In some embodiments, the peptide linker is Ser-Cys-Val-Pro-Leu-Met-Arg-Cys-Gly-Gly-Cys-Cys-Asn (SEQ ID NO: 46). In some embodiments, the peptide linker is Pro-Ser-Cys-Val-Pro-Leu-Met-Arg-Cys-Gly-Gly-Cys-Cys-Asn (SEQ ID NO: 47). In some embodiments, the peptide linker is Gly-Asp-Leu-Ile-Tyr-Arg-Asn-Gln-Lys (SEQ ID NO: 48). In some embodiments, the peptide linker is Gly₉-Pro-Ser-Cys-Val-Pro-Leu-Met-Arg-Cys-Gly-Gly-Cys-Cys-Asn (SEQ ID NO: 49).

### Chains

In some embodiments, a VEGF variant polypeptide is represented by the formula A-L-B, wherein A and B are each independently VEGF monomer subunits, L is a peptide linker. In some embodiments, L is selected from the group consisting of: GSTSGSGKSSEGKG (SEQ ID NO: 41); GSTSGSGKSSEGKGGGGGS (SEQ ID NO: 42); GGGGSGGGGSGGGG (SEQ ID NO: 43); and GGGGSGGGGSGGGGSGGGGS (SEQ ID NO: 44).

In some embodiments, the VEGF variant polypeptide is represented by the formula A-L₁-B-(L₂-A-L₁-B)ₙ-L₂-A-L₁-B, wherein A and B are each independently a VEGF monomer subunit, L₁ and L₂ are each independently a peptide linker; and n is an integer from 0 to 4. In some embodiments, L₁ is selected from the group consisting of: GSTSGSGKSSEGKG (SEQ ID NO: 41); GSTSGSGKSSEGKGGGGGS (SEQ ID NO: 42); GGGGSGGGGSGGGG (SEQ ID NO: 43); and GGGGSGGGGSGGGGSGGGGS (SEQ ID NO: 44). In some embodiments, L₂ is selected from the group consisting of: (GS)ₙ, where n=10-30; (G₂S)ₙ, where n=6-20; (G₃S)ₙ, where n=5-15; (G₄S)ₙ, where n=4-12; (G)ₙ, where n=20-60; and (S)ₙ, where n=20-60.

### Increased Half-Life

In some embodiments, a VEGF variant polypeptide has an increased plasma and/or ocular half-life as compared to the wild-type VEGF homodimer. The half-life of a protein is a measurement of protein stability and its rate of clearance and indicates the time necessary for a one-half reduction in the concentration of the protein. In some embodiments, the serum half-life of the modified VEGF molecules described herein is determined by any suitable method for measuring VEGF levels in samples from a subject over time, such as immunoassays using anti-VEGF antibodies to measure VEGF levels in serum samples taken over a period of time after administration of the modified VEGF, or by detection of labeled VEGF molecules, e.g., radiolabeled molecules, in samples taken from a subject after administration of the labeled VEGF.

Any suitable modification is used to increase the half-life of a VEGF variant polypeptide disclosed herein. In some embodiments, increased half-life is provided by the use of a Fc-fusion. In some embodiments, increased half-life is provided by the use of an albumin fusion. In some embodiments, increased half-life is provided by the use of a peptide extension such as a carboxy terminal extension peptide (CTEP) of human chorionic gonadotropin (hCG). In some embodiments, a monomer of a VEGF variant is covalently bound to a CTEP, e.g. by a peptide bond or by a heterobifunctional reagent able to form a covalent bond between the amino terminus and carboxyl terminus of a protein, including but not limited to a peptide linker. In some embodiments, a VEGF variant comprises an amino acid substitution coupled with one or more amino acid substitutions that enhance stability and increase serum half-life by eliminating one or more proteolytic cleavage sites. In some embodiments, the additional amino acid substitutions reduce proteolytic cleavage. In some embodiments, the additional amino acid substitutions prevent proteolytic cleavage. In some embodiments, increased half-life is provided by crosslinking, including but not limited to pegylation or conjugation of other appropriate chemical groups. In some embodiments, half-life is increased by increasing the number of negatively charged residues within the molecule, for instance, the number of glutamate and/or aspartate residues. In some embodiments, such alteration is accomplished by site directed mutagenesis or by an insertion of an amino acid sequence containing one or more negatively charged residues.

### Exemplary VEGF Variant Polypeptides

Disclosed herein, in certain embodiments, are VEGF variant polypeptides comprising two VEGF monomer subunits linked together by a linker, for example a peptide linker.

In some embodiments, the VEGF variant polypeptide comprises a first and a second VEGF-A monomer subunit joined by a peptide linker selected from the group consisting of: GSTSGSGKSSEGKGGGGGS (SEQ ID NO: 42), GGGGSGGGGSGGGG (SEQ ID NO: 43), and GGGGSGGGGSGGGGSGGGGS (SEQ ID NO: 44), wherein (a) the first and the second VEGF-A monomer subunits comprise any mutation selected from the group consisting of: V14A, V14I, V15A, K16R, F17L, M18R, D19G, Q22R, R23K, I29V, L325, I35V, F36L, F36S, D41N, E42K, E44G, Y45H, F47S, K48E, P49L, S50P, P53S, G58S, C60Y, D63H, D63N, D63G, I76T, M78V, M81T, M81V, R82G, H86Y, Q87R, Q89H, H90R, I91T, I91V, N100D, and K101E, and (b) loop 1, loop 2, or loop 3, or any combinations thereof, of the first and/or the second VEGF-A monomer subunit is replaced with any RGD sequence of Table 1.

In some embodiments, a VEGF variant polypeptide is a bifunctional antagonist of both VEGFR (e.g., VEGFR1 and VEGFR2) and integrin (e.g., αᵥβ₃ integrin). Exemplary bifunctional antagonist VEGF variant polypeptides include mE7I (SEQ ID NO: 75), (SEQ ID NO: 76), mJ7I (SEQ ID NO: 77), mE7I-R1null (SEQ ID NO: 78).

In some embodiments, a VEGF variant polypeptide is at least 90%, at least 95%, at least 99%, or 100% identical to a protein sequence of mE7I (SEQ ID NO: 75). In some embodiments, a VEGF variant polypeptide is at least 90%, at least 95%, at least 99%, or 100% identical to a protein sequence of mA7I (SEQ ID NO: 76). In some embodiments a VEGF variant polypeptide is at least 90%, at least 95%, at least 99%, or 100% identical to a protein sequence of mJ7I (SEQ ID NO: 77). In some embodiments, a VEGF variant polypeptide is at least 90%, at least 95%, at least 99%, or 100% identical to a protein sequence of mE7I-R1null (SEQ ID NO: 78).

### Production of VEGF Variant Polypeptides

VEGF variant polypeptides can be produced through recombinant methods or chemical synthesis methods known to the skilled artisan. In addition, functionally equivalent polypeptides may find use, where the equivalent polypeptide may contain deletions, additions or substitutions of amino acid residues that result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved. “Functionally equivalent,” as used herein, refers to a protein capable of exhibiting a substantially similar in vivo activity.

The VEGF variant polypeptides may be produced by recombinant DNA technology using techniques well known in the art. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

As an option to recombinant methods, VEGF variant polypeptides can be chemically synthesized. Such methods typically include solid-state approaches, but can also utilize solution-based chemistries and combinations or combinations of solid-state and solution approaches. Examples of solid-state methodologies for synthesizing proteins are described by Merrifield (1963) J. Am. Chem. Soc. 85:2149; and Houghten (1985) Proc. Natl. Acad. Sci., 82:5131. Fragments of polypeptides of the invention proteins can be synthesized and then joined together. Methods for conducting such reactions are described by Grant (1992) Synthetic Peptides: A User Guide, W.H. Freeman and Co., N.Y.; and in “Principles of Peptide Synthesis,” (Bodansky and Trost, ed.), Springer-Verlag, Inc. N.Y., (1993). Proteins or peptides of the invention may comprise one or more non-naturally occurring or modified amino acids. A “non-naturally occurring amino acid residue” refers to a residue, other than those naturally occurring amino acid residues listed above, which is able to covalently bind adjacent amino acid residues(s) in a polypeptide chain. Non-natural amino acids include, but are not limited to homo-lysine, homo-arginine, homo-serine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, citrulline, pentylglycine, pipecolic acid and thioproline. Modified amino acids include natural and non-natural amino acids which are chemically blocked, reversibly or irreversibly, or modified on their N-terminal amino group or their side chain groups, as for example, N-methylated D and L amino acids, side chain functional groups that are chemically modified to another functional group. For example, modified amino acids include methionine sulfoxide; methionine sulfone; aspartic acid-(beta-methyl ester), a modified amino acid of aspartic acid; N-ethylglycine, a modified amino acid of glycine; or alanine carboxamide and a modified amino acid of alanine. Additional non-natural and modified amino acids, and methods of incorporating them into proteins and peptides, are known in the art (see, e.g., Sandberg et al., (1998) J. Med. Chem. 41: 2481-91; Xie and Schultz (2005) Curr. Opin. Chem. Biol. 9: 548-554; Hodgson and Sanderson (2004) Chem. Soc. Rev. 33: 422-430.

Typically, the coding sequence for a VEGF variant polypeptide is placed under the control of a promoter that is functional in the desired host cell to produce relatively large quantities of the gene product. A wide variety of promoters is well-known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

VEGF variant polypeptides may be purified and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable binding partner immobilized on a matrix, centrifugation, ELISA, BIACore, Western blot assay, amino acid and nucleic acid sequencing, and biological activity.

### Uses

Disclosed herein, in certain embodiments, are VEGF variant polypeptides. In some embodiments, the VEGF variant polypeptides are Fc-fusions. In some embodiments, the VEGF variant polypeptides are used in methods of diagnosing and treating an angiogenic disorder.

In some embodiments, the angiogenic disorder is an angiogenesis associated eye disorder. In some embodiments, such VEGF variant polypeptides are used in treating pterygium. In some embodiments, the angiogenic disorder is ocular neovascularization, choroidal neovascularization, iris neovascularization, corneal neovascularization, retinal neovascularization, pinguecula, or pannus. In some embodiments, the angiogenic disorder is corneal neovascularization. In some embodiments, the angiogenic disorder is pinguecula. In some embodiments, the angiogenic disorder is pannus. In some embodiments, the angiogenic disorder is selected from the group consisting of diabetic retinopathy (DR), diabetic macular edema (DME), retinal detachment, posterior uveitis, and combinations thereof. In some embodiments, the angiogenic disorder is diabetic retinopathy. In some embodiments, the angiogenic disorder is macular degeneration, for example, age-related macular degeneration (AMD), particularly wet macular degeneration. In some embodiments, the angiogenic disorder is a keloid. In some embodiments, the angiogenic disorder is retinal vein occlusion. In some embodiments, the angiogenic disorder is glaucoma, cataract, partial blindness, complete blindness, myopia, myopic degeneration, deterioration of central vision, metamophopsia, color disturbances, hemorrhaging of blood vessels, or a combination thereof.

Disclosed herein, in some embodiments, are methods of treating angiogenic-associated conditions in a subject in need thereof. In some embodiments, the angiogenic-associated condition is pterygium. In some embodiments, the angiogenic-associated condition is corneal neovascularization. In some embodiments, the angiogenic-associated condition is pannus. In some embodiments, the angiogenic-associated condition corneal limbal neovascularization from, for instance, contact lens overwear. In some embodiments, the angiogenic-associated condition is pinguecula. In some embodiments, the methods comprise administration of a polypeptide disclosed herein to the subject.

Pterygium (also known as “Surfer's Eye”) is a benign vascular growth across the conjunctival and corneal surface of the eye. Pterygium is characterized by a wedge-shaped, highly vascular, fleshy growth that originates on the conjunctiva and that, in some instances, spreads to the corneal limbus and beyond. The pterygium commonly grows from the nasal side of the sclera and is usually present in the palpebral fissure. It is associated with and thought to be caused by ultraviolet-light exposure (e.g., sunlight), low humidity, wind and dust. In some instances, the pterygium is preceded with scleral trauma around the Palpebral comissure. In some instances, the predominance of pterygia on the nasal side is a result of the sun's rays passing laterally through the cornea, where it undergoes refraction and becomes focused on the limbic area. Sunlight passes unobstructed from the lateral side of the eye, focusing on the medial limbus after passing through the cornea. On the contralateral (medial) side, however, the shadow of the nose medially reduces the intensity of sunlight focused on the lateral/temporal limbus.

Pterygium in the conjunctiva is characterized by elastic degeneration of collagen (actinic elastosis) and fibrovascular proliferation. Pterygium generally exhibits neovascularization, remodeling of the extracellular matrix (ECM), and proliferating fibroblasts (FBs). It has an advancing portion called the head of the pterygium, which is connected to the main body of the pterygium by the neck. In some instances, a line of iron deposition is seen adjacent to the head of the pterygium called Stockers line. In some instances, the location of the line gives an indication of the pattern of growth.

Pterygium is composed of several segments: Fuchs' Patches (minute gray blemishes that disperse near the pterygium head), Stockers Line (a brownish line composed of iron deposits), Hood (fibrous nonvascular portion of the pterygium), Head (apex of the pterygium, typically raised and highly vascular), Body (fleshy elevated portion congested with tortuous vessels), Superior Edge (upper edge of the triangular or wing-shaped portion of the pterygium), Inferior Edge (lower edge of the triangular or wing-shaped portion of the pterygium).

In some instances, because pterygium is caused by excessive sun or wind exposure, protective sunglasses with side shields or wide brimmed hats and application of artificial tears to the eyes aids in preventing pterygium formation or prevent further growth.

Additional angiogenic-associated conditions for treatment with the polypeptides disclosed herein include pinguecula, pannus, and corneal neovascularization. Pinguecula is conjunctival degeneration of the eye. Individuals with pinguecula present with yellow-white deposit on the conjunctiva adjacent to the limbus. Histologically, the disorder is characterized by degeneration of the collagen fibers of the conjunctiva stroma with thinning of the overlying epithelium and occasional calcification. Pannus is an abnormal layer of blood vessels into the peripheral cornea. Corneal neovascularization is the excessive ingrowth of blood vessels from the limbal vascular plexus into the cornea often associated with inflammation of or trauma to the cornea.

Treatment with the polypeptides of the present invention can be combined with conventional treatment for pterygium, which include, but are not limited to surgical removal and/or irradiation, conjunctival autografting, amniotic membrane transplantation, or administration of a therapeutic agent. If pterygium recurs after surgery, or is thought to be vision threatening, strontium (⁹⁰Sr) plaque therapy may be used. Conjunctival auto-grafting is an invasive surgical technique for pterygium growth removal. Amniotic membrane transplantation is also used for pterygium growth removal. Other therapeutic agents for the treatment of pterygium include but are not limited to mitomycin C (MMC), 5-fluorouracil (5-FU), loteprednol etabonate (LE), oral doxycycline, dipyridamole, and dobesilate.

In some embodiments, the angiogeneic disorder is a cancer. In some embodiments, the cancer is prostate cancer, breast cancer, lung cancer, esophageal cancer, colon cancer, rectal cancer, liver cancer, urinary tract cancer (e.g., bladder cancer), kidney cancer, lung cancer (e.g., non-small cell lung cancer), ovarian cancer, cervical cancer, endometrial cancer, pancreatic cancer, stomach cancer, thyroid cancer, skin cancer (e.g., melanoma), hematopoietic cancers of lymphoid or myeloid lineage, head and neck cancer, nasopharyngeal carcinoma (NPC), glioblastoma, teratocarcinoma, neuroblastoma, adenocarcinoma, cancers of mesenchymal origin such as a fibrosarcoma or rhabdomyosarcoma, soft tissue sarcoma and carcinoma, choriocarcinioma, hepatoblastoma, Karposi's sarcoma or Wilm's tumor.

In some embodiments, the angiogenic disorder is an inflammatory disorder. In some embodiments, the inflammatory disorder is inflammatory arthritis, osteoarthritis, psoriasis, chronic inflammation, irritable bowel disease, lung inflammation or asthma.

In some embodiments, the angiogenic disorder is an autoimmune disorder. In some embodiments, the autoimmune disease is rheumatoid arthritis, multiple sclerosis, or systemic lupus erythematosus.

Other angiogenic disorders include atherosclerosis, retrolentral fibroplasia, thyroid hyperplasias (including grave's disease), nephrotic syndrome, preclampasia, ascites, pericardial effusion (such as associated with pericarditis) and pleural effusion.

### Combination Therapy

In some embodiments, the VEGF variant polypeptide is administered to the individual in combination with an additional therapeutic agent. In some embodiments, the additional therapeutic is an inhibitor of a vascular endothelial growth factor (VEGF), a platetlet-derived growth factor (PDGF), an angiotensin (ANG), or a fibroblast growth factor (FGF), and associated receptors. In some embodiments, the additional therapeutic is an inhibitor of a matrix metalloprotinease (MMP), prostate-specific membrane antigen (PSMA). In some embodiments, the additional therapeutic is selected from the group consisting of an antibody, polypeptide, nucleotide, a small molecule, and combinations thereof. In some embodiments, the additional therapeutic agent is selected from the group consisting of: mitomycin C (MMC), 5-fluorouracil (5-FU), loteprednol etabonate (LE), oral doxycycline, dipyridamole, and dobesilate. In some embodiments, the additional therapeutic agent is an anti-inflammatory steroid. In some embodiments, the additional therapeutic agent is non-steroidal anti-inflammatory agent. In some embodiments, the additional therapeutic agent is an antibody or small molecule inhibitor of VEGF signaling. In some embodiments, the additional therapeutic agent binds, traps, scavenges or otherwise deters the effect of VEGF that has already been produced.

In some embodiments, the additional therapeutic agent is a chemotherapeutic agent. In some embodiments, the additional therapweutic agent is selected from: alkylating agents, e.g. Cisplatin, Cyclophosphamide, Altretamine; DNA strand-breakage agents, such as Bleomycin; DNA topoisomerase II inhibitors, including intercalators, such as Amsacrine, Dactinomycin, Daunorubicin, Doxorubicin, Idarubicin, and Mitoxantrone; nonintercalating topoisomerase II inhibitors such as, Etoposide and Teniposide; DNA minor groove binder Plicamycin; alkylating agents, including nitrogen mustards such as Chlorambucil, Cyclophosphamide, Isofamide, Mechlorethamine, Melphalan, Uracil mustard; aziridines such as Thiotepa; methanesulfonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine; antimetabolites, including folate antagonists such as Methotrexate and trimetrexate; pyrimidine antagonists, such as Fluorouracil, Fluorodeoxyuridine, CB3717, Azacytidine, Cytarabine; Floxuridine purine antagonists including Mercaptopurine, 6-Thioguanine, Fludarabine, Pentostatin; sugar modified analogs include Cyctrabine, Fludarabine; ribonucleotide reductase inhibitors including hydroxyurea; Tubulin interactive agents including Vincristine Vinblastine, and Paclitaxel; adrenal corticosteroids such as Prednisone, Dexamethasone, Methylprednisolone, and Prodnisolone; hormonal blocking agents including estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone estrogens, conjugated estrogens and Ethinyl Estradiol and Diethylstilbesterol, Chlorotrianisene and Idenestrol.

In some embodiments, a VEGF variant polypeptide and the additional therapeutic agent are administered in a unified dosage form or in separate dosage forms. In some embodiments, the methods comprise administration of a VEGF variant polypeptide disclosed herein in combination with a therapeutic procedure. Procedures that provide additional or synergistic benefit include, but are not limited to irradiation (e.g. 90Sr therapy), conjunctival autografting or amniotic membrane transplantation, or surgery.

By way of example only, if one of the side effects experienced by an individual upon receiving one of the VEGF variant polypeptides described herein is nausea, then it is appropriate to administer an anti-nausea agent in combination with the initial therapeutic agent. Or, by way of example only, the therapeutic effectiveness of one of the therapeutic agents described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by an individual is increased by administering one of the therapeutic agents described herein with another therapeutic agent (which also includes a therapeutic regimen) that also has therapeutic benefit. In any case, regardless of the disease or disorder being treated, the overall benefit experienced by the patient is simply additive of the two therapeutic agents or in other embodiments, the patient experiences a synergistic benefit.

The particular choice of agents used will depend upon the diagnosis of the attending physicians and their judgment of the condition of the patient and the appropriate treatment protocol. The agents are optionally administered concurrently (e.g., simultaneously, essentially simultaneously or within the same treatment protocol) or sequentially, depending upon the nature of the disorder, the condition of the patient, and the actual choice of agents used. The determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

### Pharmaceutical Formulations

In some embodiments, while it is possible to use an agent disclosed herein for therapy as is, it is preferable to administer the agent as a pharmaceutical formulation, e.g., in a mixture with a suitable pharmaceutical excipient, diluent, or carrier selected with regard to the intended route of administration and standard pharmaceutical practice. Pharmaceutical formulations include at least one active compound, in association with a pharmaceutically acceptable excipient, diluent, and/or carrier. In some embodiments, the dose and the administration frequency are adjusted based on the judgment of the treating physician, for example taking into account the clinical signs, pathological signs and clinical and subclinical symptoms of a disease of the conditions treated with the present methods, as well as the patient's clinical history. For example, higher doses, increased frequency of administration, or a longer duration of treatment are indicated when a patient is showing symptoms of pterygium or keloid recurrence (e.g., blood vessel growth), or if the patient has a history of previous pterygium or keloid recurrence.

Formulations of polypeptides find use in diagnosis and therapy. In some embodiments, the formulation comprises one, two or more polypeptides or agents. In some embodiments, the therapeutic formulation is administered in combination with other methods of treatment, e.g. chemotherapy, radiation therapy, surgery, and the like.

In some embodiments, formulations are optimized for retention and stabilization at a targeted site. Stabilization techniques include enhancing the size of the polypeptide, by cross-linking, multimerizing, or linking to groups such as polyethylene glycol, polyacrylamide, neutral protein carriers, Fc-fusions etc. in order to achieve an increase in molecular weight. Other strategies for increasing retention include the entrapment of the polypeptide in a biodegradable or bioerodible implant or biogel, or by a non bioerodible polymeric reservoir. Still other strategies for increasing retention include the chemical entrapment of the polypeptide in a biodegradable or bioerodible implant or biogel, or by a non bioerodible polymeric reservoir, with slow release of the polypeptide by degradation of the chemical linkage to the reservoir. The rate of release of the therapeutically active agent is controlled by the rate of transport through the polymeric matrix, and the biodegradation of the implant. The transport of polypeptide through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. In some embodiments, implants include, e.g., particles, sheets, patches, plaques, fibers, or microcapsules and are any size or shape compatible with the selected insertion site.

In some embodiments, pharmaceutical compositions include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In some embodiments, the pharmaceutical composition or formulation includes other carriers, adjuvants, or non-toxic, nontherapeutic, non-immunogenic stabilizers, excipients and the like. In some embodiments, the compositions also include additional substances to approximate physiological conditions, such as pH adjusting and buffering agents, toxicity adjusting agents, wetting agents and detergents.

In some embodiments, the composition includes any of a variety of stabilizing agents, such as an antioxidant, for example. In some embodiments, the peptide is complexed with various well-known compounds that enhance the in vivo stability of the peptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. In some embodiments, the peptides of a composition are complexed with molecules that enhance their in vivo attributes. Such molecules include, for example, carbohydrates, polyamines, amino acids, other peptides, ions (e.g., sodium, potassium, calcium, magnesium, manganese), and lipids.

In some embodiments, the pharmaceutical compositions are administered for prophylactic and/or therapeutic treatments. In some embodiments, toxicity and therapeutic efficacy of the active ingredient are determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

In some embodiments, the data obtained from cell culture and/or animal studies are used in formulating a range of dosages for humans. The dosage of the active ingredient typically lies within a range of circulating concentrations that include the ED₅₀ with low toxicity. In some embodiments, the dosage varies within this range depending upon the dosage form employed and the route of administration utilized.

The pharmaceutical compositions described herein are administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

Formulations suitable for parenteral administration, such as, for example, by intravenous, intralesional, intramuscular, intradermal, intraperitoneal, and subcutaneous routes, include aqueous and non-aqueous, isotonic sterile injection solutions, which in some embodiments contain antioxidants, buffers, bacteriostats, and solutes that render the formulation isotonic with the blood of the intended recipient, and aqueous and non-aqueous sterile suspensions that in some embodiments include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, in some embodiments, the resulting product is typically substantially free of any potentially toxic agents, particularly any endotoxins, which are present during the synthesis or purification process. Compositions for parental administration are also sterile, substantially isotonic and made under GMP conditions.

In some embodiments, are ophthalmic formulations for pterygium treatment. In some embodiments a VEGF variant polypeptide is provided as an ophthalmic formulation for treating pterygium. In some embodiments, the ophthalmic formulation comprises any preparations for conjunctival topical use to be applied to conjunctival mucosa. In some embodiments, the ophthalmic formulation is a liquid preparation (e.g., aqueous or oily solutions or suspensions), or solid preparation (e.g., ointments, powders) for the treatment of an ocular condition, (e.g., pterygium). In some embodiments, the ophthalmic formulation is an ointment. In some embodiments, the ophthalmic formulation is a cream. In some embodiments, other substances are present as excipients in the formulation including anti-oxidant and visco-elastic compounds or vehicles, preservatives, buffer solutions, osmolar and emulsifying substances (or tensioactives).

In some embodiments, the composition comprises one or more excipients such as polyethylene glycol or vaseline and nonionic emulsifying substances (or tensioactives) (such as polysorbate) that could be used for a better tolerability. Ophthalmic formulations for topical use are preferably prepared with a tolerable pH, generally in the range of 6.4-7.8, sterile and devoid of exogenous particles and with a tear-isotonic osmotic pressure around 300 mOsm/L or anywhere between about 200 and about 350 mOsm/L.

Surgical operation for treating pterygium consists of the detachment and removal of pterygium head, followed by conjunctival suture leaving an ample portion of bare sclera or attaching the tissue up to the corneoscleral limbus. In some embodiments, a conjunctival reconstruction is necessary through the sliding of the tissue or even the autologous transplant of conjunctiva. After this type of procedure, the most common post-surgery complications include infection, conjunctival cysts or adherent scars limiting ocular movements. After surgery treatment, it remains possible to develop relapse of more aggressive forms with a higher proliferation index, with a prevalence that ranges between 10-80% of cases. Thus, in some embodiments, the utilization of eye drops according to the invention is advantageous. In some embodiments, it prevents or delays pterygium growth and reduces the necessity for surgical interventions and post-surgery complications.

In some embodiments, the ophthalmic compound is formulated as eye drops, gel, cream or ointment in aqueous or hydro-soluble solvents (e.g., alcohol). Exemplary aqueous solvents include phosphate or citrate phosphate or TRIS buffer, or buffers containing histidine, tricine, lysine, glycine, and/or serine. In some embodiments, solvents are adjusted to the right physiological pH with an acid or basic component. In some embodiments, agents increasing solubility, preservatives, visco-elastic substances (preferably in the range 0.1-10% v/v) (such as hyaluronic acid, polyethylene glycol, mixtures of polyethylene glycol with fatty acids), or celluloses (like hydroxyl-propyl-methyl cellulose) are present. Potentially, also anti-oxidant substances, like ascorbic acid in the range 1-15% v/v and chelating agents like EDTA, are contained in the formulation.

In determining the effective amount of a polypeptide, the route of administration, the kinetics of the release system (e.g., pill, gel or other matrix), and the potency of the agent are considered so as to achieve the desired effect with minimal adverse side effects. The dosage of a polypeptide of the invention is adjusted according to the potency and/or efficacy relative to a VEGF or PDGF antagonist. In some embodiments, a dose is in the range of about 0.001 μg to 100 mg, given 1 to 20 times daily, and be up to a total daily dose of about 0.01 μg to 100 mg. In some embodiments, if applied topically, for the purpose of a systemic effect, the patch or cream is designed to provide for systemic delivery of a dose in the range of about 0.01 μg to 100 mg. In some embodiments, if injected for the purpose of a systemic effect, the matrix in which the polypeptide is administered is designed to provide for a systemic delivery of a dose in the range of about 0.001 μg to 1 mg. If injected for the purpose of a local effect, the matrix is designed to release locally an amount of VEGF variant polypeptide in the range of about 0.001 μg to 100 mg.

In some embodiments, while it is possible to use an agent disclosed herein for therapy as is, it is preferable to administer the agent as a pharmaceutical formulation, e.g., in a mixture with a suitable pharmaceutical excipient, diluent, or carrier selected with regard to the intended route of administration and standard pharmaceutical practice. Pharmaceutical formulations include at least one active compound, in association with a pharmaceutically acceptable excipient, diluent, and/or carrier. In some embodiments, the dose and the administration frequency are adjusted based on the judgment of the treating physician, for example taking into account the clinical signs, pathological signs and clinical and subclinical symptoms of a disease of the conditions treated with the present methods, as well as the patient's clinical history. For example, higher doses, increased frequency of administration, or a longer duration of treatment are indicated when a patient is showing symptoms of pterygium or keloid recurrence (e.g., blood vessel growth), or if the patient has a history of previous pterygium or keloid recurrence.

Formulations of polypeptides find use in diagnosis and therapy. In some embodiments, the formulation comprises one, two or more polypeptides or agents. In some embodiments, the therapeutic formulation is administered in combination with other methods of treatment, e.g. chemotherapy, radiation therapy, surgery, and the like.

In some embodiments, formulations are optimized for retention and stabilization at a targeted site. Stabilization techniques include enhancing the size of the polypeptide, by cross-linking, multimerizing, or linking to groups such as polyethylene glycol, polyacrylamide, neutral protein carriers, Fc-fusions etc. in order to achieve an increase in molecular weight. Other strategies for increasing retention include the entrapment of the polypeptide in a biodegradable or bioerodible implant or biogel, or by a non bioerodible polymeric reservoir. The rate of release of the therapeutically active agent is controlled by the rate of transport through the polymeric matrix, and the biodegradation of the implant. The transport of polypeptide through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. In some embodiments, implants include, e.g., particles, sheets, patches, plaques, fibers, or microcapsules and are any size or shape compatible with the selected insertion site.

In some embodiments, ophthalmic compositions are formulated for pterygium treatment. In some embodiments, the ophthalmic formulation comprises any preparations for conjunctival topical use to be applied to conjunctival mucosa. In some embodiments, the ophthalmic formulation is a liquid preparation (e.g., aqueous or oily solutions or suspensions), or solid preparation (e.g., ointments, powders) for the treatment of an ocular condition, (e.g., pterygium). In some embodiments, the ophthalmic formulation is an ointment. In some embodiments, the ophthalmic formulation is a cream. In some embodiments, other substances are present as excipients in the formulation including anti-oxidant and visco-elastic compounds or vehicles, preservatives, buffer solutions, osmolar and emulsifying substances (or tensioactives).

In some embodiments, the composition comprises one or more excipients such as polyethylene glycol or vaseline and nonionic emulsifying substances (or tensioactives) (such as polysorbate) that could be used for a better tolerability. Ophthalmic formulations for topical use are preferably prepared with a tolerable pH, generally in the range of 6.4-7.8, sterile and devoid of exogenous particles and with a tear-isotonic osmotic pressure around 300 mOsm/L or anywhere between about 200 and about 350 mOsm/L. In some embodiments, the ophthalmic compound is formulated as eye drops, gel, cream or ointment in aqueous or hydro-soluble solvents (e.g., alcohol). Exemplary aqueous solvents include phosphate or citrate phosphate or TRIS buffer, or buffers containing histidine, tricine, lysine, glycine, and/or serine. In some embodiments, solvents are adjusted to the right physiological pH with an acid or basic component. In some embodiments, agents increasing solubility, preservatives, visco-elastic substances (preferably in the range 0.1-10% v/v) (such as hyaluronic acid, polyethylene glycol, mixtures of polyethylene glycol with fatty acids), or celluloses (like hydroxyl-propyl-methyl cellulose) are present. Potentially, also anti-oxidant substances, like ascorbic acid in the range 1-15% v/v and chelating agents like EDTA, are contained in the formulation.

Disclosed herein, in some embodiments, are methods of treating an ocular disorder, for example pterygium, in a subject in need thereof. In some embodiments, the methods comprise administration of a polypeptide of the present invention and an additional therapeutic agent to the subject. In some embodiments, the additional therapeutic agent is an inhibitor of a vascular endothelial growth factor (VEGF), a platelet-derived growth factor (PDGF), a fibroblast growth factor (FGF), or an angiotensin (ANG), and associated receptors. In some embodiments, the additional therapeutic agent is an inhibitor of an integrin, or an inhibitor of a matrix metalloproteinase (MMP), or prostate specific membrane antigen (PSMA). In some embodiments, the additional therapeutic is selected from the group consisting of an antibody, polypeptide, nucleotide, a small molecule, and combinations thereof. In some embodiments, the additional therapeutic agent is selected from the group consisting of: mitomycin C (MMC), 5-fluorouracil (5-FU), loteprednol etabonate (LE), oral doxycycline, dipyridamole, and dobesilate. In some embodiments, the additional therapeutic agent is an anti-inflammatory steroid. In some embodiments, the additional therapeutic agent is non-steroidal anti-inflammatory agent. In some embodiments, the additional therapeutic agent is an antibody or small molecule inhibitor of VEGF signaling. In some embodiments, the additional therapeutic agent binds, traps, scavenges or otherwise deters the effect of VEGF that has already been produced.

In some embodiments, the polypeptide of the present invention and the additional therapeutic agent are administered in a unified dosage form or in separate dosage forms. In some embodiments, the methods comprise administration of a polypeptide disclosed herein in combination with a therapeutic procedure. Procedures that provide additional or synergistic benefit include, but are not limited to irradiation (e.g. ⁹⁰Sr therapy), conjunctival autografting or amniotic membrane transplantation, or surgery.

By way of example only, the therapeutic effectiveness of one of the therapeutic agents described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by an individual is increased by administering one of the therapeutic agents described herein with another therapeutic agent (which also includes a therapeutic regimen) that also has therapeutic benefit. In any case, regardless of the disease or disorder being treated, the overall benefit experienced by the patient is simply additive of the two therapeutic agents or in other embodiments, the patient experiences a synergistic benefit.

The particular choice of agents used will depend upon the diagnosis of the attending physicians and their judgment of the condition of the patient and the appropriate treatment protocol. The agents are optionally administered concurrently (e.g., simultaneously, essentially simultaneously or within the same treatment protocol) or sequentially, depending upon the nature of the disorder, the condition of the patient, and the actual choice of agents used. The determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

In another aspect, a pharmaceutical composition comprising a polypeptide of the present invention is incorporated into an ophthalmic device that comprises a biodegradable material, and the device is implanted into a subject to provide a long-term (e.g., longer than about 1 week, or longer than about 1, 2, 3, 4, 5, or 6 months) treatment of the ocular condition, such as pterygium. Such a device is implanted by a skilled physician in the subject's ocular or periocular tissue.

The methods of treating conditions with a pharmaceutical composition comprising a polypeptide described herein offer advantages both over surgical methods of treatment and over existing biologic agents. No non-surgical intervention exists for early or advanced pterygium. Furthermore, even if entirely successful in removal of the vascular and fibrous tissue components, surgery cannot prevent the recurrence of pterygium. Repeat invasive surgeries for excision of pterygium carry significant risks. Hence, a pharmaceutical composition comprising a polypeptide that controls the growth of existing pterygium and/or prevent the recurrence of pterygium post-surgical excision are advantageous. In some embodiments, a pharmaceutical composition comprising a polypeptide of the present invention is administered during and/or immediately after surgery, such as by intralesional injection, subconjunctival injection, or other direct application to or near the pterygium site. In some embodiments, a course of treatment combines elements of the above, such as administration during and/or after surgery by injection or other technique, plus at-home (out-of-office) administered eye drops or other means of topical administration in the days, weeks, and/or months after surgery. In some embodiments, a pharmaceutical composition comprising a polypeptide of the present invention is used to treat a condition instead of surgery, to halt progression or induce regression of the condition. If the pharmaceutical composition comprising a polypeptide of the present invention is shown to be particularly effective, then patients and physicians, who might have otherwise opted for pterygium surgery, might opt for treatment with a pharmaceutical composition alone instead of surgery, to avoid the cost, time, pain, and risk of surgery. Other patient classes that would benefit from a pharmaceutical composition without surgery include those that don't qualify for surgery, those that can't afford surgery, and those who qualify for but choose to not undergo surgery. Second, a pharmaceutical composition could be used during and/or after surgery, to prevent recurrence, particularly because of unacceptably high recurrence rates in past and present techniques, or the need for very complex forms of surgery that include ocular tissue transplantation or transfer.

In some embodiments, a method of treatment involves professional intervention combined with administration of a pharmaceutical composition comprising a polypeptide of the present invention. For example, in some embodiments, a method of treatment first involves debridement of the surface layer of a pterygium such as the epithelium or superficial fibroblastic layer, followed by administration of a pharmaceutical composition comprising a polypeptide of the present invention. The administration can be topical or intralesional. In some embodiments, debridement is a simpler, less expensive, shorter, and lower-risk intervention that enables or enhances the effect of a pharmaceutical composition, such as by exposing endothelial cells, fibroblasts, or other cells to the anti-angiogenic, anti-growth, and/or anti-migratory effects of the polypeptide or otherwise enhancing their penetration into the lesion.

Existing biologics target only a subset of ligand-receptor interactions that mediate angiogenesis which inherently limits their efficacy. In some embodiments, the polypeptides described herein target multiple receptors and exhibit superior efficacy compared to agents that target fewer, or a single target. Furthermore, the polypeptide compositions utilize a soluble growth factor scaffold, and are significantly smaller in size (25 kDa) when compared to existing biologics (50-150 kDa) which are either antibodies, antibody fragments or receptor extra-cellular domains fused to antibody Fc domains. Accordingly, whereas the large size of the existing biologics necessitates delivery via injection (subconjunctival), in some embodiments, a pharmaceutical composition comprising a polypeptide described herein is administered topically. This represents a significant reduction in patient compliance burden and the cost of therapy.

Ideally, a treatment for pterygium, whether post-surgery, to reduce rates of recurrence, or instead of surgery, to halt progression or induce regression, would be easily and safely administered, such as topical eye drops or other similar formulations such as viscous gels, or ointments. A preferred method of treatment is a topical eye drop, self-administered as infrequent as once per course of treatment or once per month. Less preferred, but still very satisfactory, is more frequent self-administered topical formulations, since that still avoids the time, cost, pain, and risk of injections. For example, eye drops, gels or ointments applied out-of-office once per week, twice per week, once per day, or twice per day, or three times per day or four times per day.

### Routes of Administration

In some embodiments, a pharmaceutical composition comprising a VEGF variant polypeptide disclosed herein is administered topically or parenterally, or by any other suitable methods known in the art.

In some embodiments, a pharmaceutical composition comprising a VEGF variant polypeptide is formulated as an ophthalmic topical formulation; an ophthalmic injectable formulation; or for use with an ophthalmic implant. In some embodiments a pharmaceutical composition comprising a VEGF variant polypeptide is administered via subconjunctival injection or intralesional injection. In some embodiments a pharmaceutical composition comprising a VEGF variant polypeptide is administered topically to the eye.

The term “parenteral” includes injection or deposition or sustained release via vehicles or devices (e.g., intravenous, subconjunctival, subtenon, episcleral, intrascleral, subscleral, intraperitoneal, epidural, intrathecal, intramuscular, intraluminal, intratracheal, epidermal, intradermal, subdermal or subcutaneous). Moreover, in some embodiments, the different agents administered in the combination therapy disclosed herein are administered by different routes. For example, in some embodiments, a VEGF variant polypeptide disclosed herein is injected into the eye or skin, or applied topically. An anti-inflammatory steroid and/or or NSAID is administered systemically (e.g., by injection), orally, and/or topically, e.g., to the eye or skin. Non-limiting examples of methods of administration include subcutaneous injection, intravenous injection, and infusion. In some embodiments, the administration is subcutaneous administration. In some embodiments, the administration is via any route practical, such as, for example, an intravenous injection, a bolus injection, infusion over 5 minutes to about 5 hours, a pill, a capsule, transdermal patch, buccal delivery, and the like, or combination thereof.

In some embodiments, a pharmaceutical composition comprising a VEGF variant polypeptide as disclosed herein is incorporated into a formulation for topical administration, systemic administration, periocular injection, or intravitreal injection. In some embodiments, an injectable intravitreal formulation comprises a carrier that provides a sustained-release of the active ingredients, such as for a period longer than about 1 week (or longer than about 1, 2, 3, 4, 5, or 6 months). In some embodiments, the sustained-release formulation desirably comprises a carrier that is insoluble or only sparingly soluble in the vitreous. In some embodiments, such a carrier is an oil-based liquid, emulsion, gel, or semisolid. Non-limiting examples of oil-based liquids include castor oil, peanut oil, olive oil, coconut oil, sesame oil, cottonseed oil, corn oil, sunflower oil, fish-liver oil, arachis oil, and liquid paraffin.

In one embodiment, a pharmaceutical composition comprising a VEGF variant polypeptide is injected intravitreally, for example through the pars plana of the ciliary body, to treat or prevent pterygium or progression thereof using a fine-gauge needle, such as 25-34 gauge.

In another aspect, a pharmaceutical composition comprising a VEGF variant polypeptide is incorporated into an ophthalmic device that comprises a biodegradable material, and the device is implanted into a subject to provide a long-term (e.g., longer than about 1 week, or longer than about 1, 2, 3, 4, 5, or 6 months) treatment of the ocular condition. Such a device is implanted by a skilled physician in the subject's ocular or periocular tissue.

In some embodiments, a method of treatment involves professional intervention combined with administration of a pharmaceutical composition comprising a VEGF variant polypeptide.

The methods of treating conditions with a pharmaceutical composition comprising a VEGF variant polypeptide described herein offer advantages both over conventional therapies. For example, with respect to pterygium, no non-surgical intervention exists for early or advanced pterygium. Furthermore, even if entirely successful in removal of the vascular and fibrous tissue components, surgery cannot prevent the recurrence of pterygium. Repeat invasive surgeries for excision of pterygium carry significant risks. Hence, a pharmaceutical composition comprising a VEGF variant polypeptide that controls the growth of existing pterygium and/or prevent the recurrence of pterygium post-surgical excision are advantageous.

In some embodiments, a pharmaceutical composition comprising a VEGF variant polypeptide or a Fc-VEGF variant polypeptide fusion is administered during and/or immediately after surgery to treat an angiogenic disorder, such as by intralesional injection, subconjunctival injection, or other direct application to or near the surgical site. In some embodiments, a course of treatment combines elements of the above, such as administration during and/or after surgery by injection or other technique, plus at-home (out-of-office) administered eye drops or other means of topical administration in the days, weeks, and/or months after surgery.

In some embodiments, a pharmaceutical composition comprising a VEGF variant polypeptide or a Fc-VEGF variant polypeptide fusion is used to treat a condition instead of surgery, to halt progression or induce regression of the condition. If the pharmaceutical composition comprising a VEGF variant polypeptide or a Fc-VEGF variant polypeptide fusion is shown to be particularly effective, then patients and physicians, who might have otherwise opted for surgery, might opt for treatment with a pharmaceutical composition comprising a VEGF variant polypeptide or a Fc-VEGF variant polypeptide fusion alone instead of surgery, to avoid the cost, time, pain, and risk of surgery. Other patient classes that would benefit from a pharmaceutical composition comprising a VEGF variant polypeptide or a Fc-VEGF variant polypeptide fusion without surgery include those that don't qualify for surgery, those that can't afford surgery, and those who qualify for but choose to not undergo surgery. Second, a pharmaceutical composition comprising a VEGF variant polypeptide or a Fc-VEGF variant polypeptide fusion could be used during and/or after surgery, to prevent recurrence, particularly because of unacceptably high recurrence rates in past and present techniques, or the need for very complex forms of surgery that include ocular tissue transplantation or transfer.

In some embodiments, a method of treatment involves professional intervention combined with administration of a pharmaceutical composition comprising a VEGF variant polypeptide or a Fc-VEGF variant polypeptide fusion. For example, in some embodiments, a method of treatment first involves a surgical intervention, such a debridment for pterygium, followed by administration of a pharmaceutical composition comprising a VEGF variant polypeptide or a Fc-VEGF variant polypeptide fusion. In some embodiments, surgical intervention enables or enhances the effect of a pharmaceutical composition comprising a VEGF variant polypeptide or a Fc-VEGF variant polypeptide fusion, such as by exposing endothelial cells, fibroblasts, or other cells to the anti-angiogenic, anti-growth, and/or anti-migratory effects of the VEGF variant polypeptide or the Fc-VEGF variant polypeptide fusion or otherwise enhancing their penetration into.

Existing anti-VEGF treatments are non-ideal due to their method of administration. Existing biologics target only a subset of ligand-receptor interactions that mediate angiogenesis which inherently limits their efficacy. In some embodiments, the VEGF variant polypeptides and Fc-VEGF variant polypeptide fusions described herein target multiple receptors and exhibit superior efficacy compared to agents that target fewer, or a single target. Furthermore, the VEGF variant polypeptide and Fc-VEGF variant polypeptide fusion compositions utilize a soluble growth factor scaffold, (VEGF itself) and are significantly smaller in size (25 kDa) when compared to existing biologics (50-150 kDa) which are either antibodies, antibody fragments or receptor extra-cellular domains fused to antibody Fc domains. Accordingly, whereas the large size of the existing biologics necessitates delivery via injection (subconjunctival), in some embodiments, a pharmaceutical composition comprising a VEGF variant polypeptide or a Fc-VEGF variant polypeptide fusion described herein is administered topically. This represents a significant reduction in patient compliance burden and the cost of therapy.

In some embodiments, the compostions disclosed herein are administered as topical eye drops or other similar formulations such as viscous gels, or ointments. In some embodiments, a topical eye drop is self-administered as infrequent as once per course of treatment or once per month. In some embodiments, a topical eye drop is administered once per week, twice per week, once per day, or twice per day, or three times per day or four times per day.

### Dosing and Treatment Regimens

In some embodiments, the dose of a pharmaceutical composition comprising a VEGF variant polypeptide administered to a subject, particularly a human, is sufficient to effect a therapeutic reduction in angiogenesis in the subject over a reasonable time frame. In some embodiments, the dose is determined by the potency of the particular peptide employed and the condition of the subject, as well as the body weight of the subject to be treated. The size of the dose also will be determined by the existence, nature, and extent of any adverse side-effects that might accompany the administration of a particular compound.

It will be appreciated that the amount of a pharmaceutical composition comprising a VEGF variant polypeptide disclosed herein required for use in treatment will vary with the route of administration, the nature of the condition for which treatment is required, and the age, body weight and condition of the patient, and will be ultimately at the discretion of the attendant physician or veterinarian. Compositions will typically contain an effective amount of the active agent(s), alone or in combination. In some embodiments, preliminary doses are determined according to animal tests, and the scaling of dosages for human administration are performed according to art-accepted practices.

In determining the effective amount of a VEGF variant polypeptide, the route of administration, the kinetics of the release system (e.g., pill, gel or other matrix), and the potency of the antagonist are considered so as to achieve the desired effect with minimal adverse side effects.

The dosage of a VEGF variant polypeptide is adjusted according to the potency and/or efficacy relative to a VEGF antagonist. In some embodiments, a dose is in the range of about 0.001 μg to 100 mg, given 1 to 20 times daily, and be up to a total daily dose of about 0.01 μg to 100 mg. In some embodiments, if applied topically, for the purpose of a systemic effect, the patch or cream is designed to provide for systemic delivery of a dose in the range of about 0.01 μg to 100 mg. In some embodiments, if injected for the purpose of a systemic effect, the matrix in which the VEGF variant polypeptide is administered is designed to provide for a systemic delivery of a dose in the range of about 0.001 μg to 1 mg. If injected for the purpose of a local effect, the matrix is designed to release locally an amount of VEGF variant polypeptide in the range of about 0.001 μg to 100 mg.

In some embodiments, dosage ranges for a pharmaceutical composition comprising a VEGF variant polypeptide described herein are determined by the ordinarily skilled artisan, and are, e.g., first be determined in animal models for determining dosage, safety and efficacy according to standard methods known in the art.

In some embodiments, a therapeutically effective amount of a pharmaceutical composition comprising a VEGF variant polypeptide is expressed as mg of the VEGF variant polypeptide per kg of subject body mass. In some embodiments, a therapeutically effective amount is 1-1,000 mg/kg, 1-500 mg/kg, 1-250 mg/kg, 1-100 mg/kg, 1-50 mg/kg, 1-25 mg/kg, or 1-10 mg/kg. In some embodiments, an effective amount is 5 mg/kg, 10 mg/kg, 25 mg/kg, 50 mg/kg, 75 mg/kg, 100 mg/kg, 150 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1,000 mg/kg, about 5 mg/kg, about 10 mg/kg, about 25 mg/kg, about 50 mg/kg, about 75 mg/kg, about 100 mg/kg, about 150 mg/kg, about 200 mg/kg, about 250 mg/kg, about 300 mg/kg, about 400 mg/kg, about 500 mg/kg, about 600 mg/kg, about 700 mg/kg, about 800 mg/kg, about 900 mg/kg, or about 1,000 mg/kg.

In some embodiments, a therapeutically effective amount is expressed as mg of the compound per square meter of subject body area. In some embodiments, a pharmaceutical composition comprising a VEGF variant polypeptide is administered subcutaneously in a range of doses, for example 1 to 1500 mg (0.6 to 938 mg/m²), or 2 to 800 mg (1.25 to 500 mg/m²), or 5 to 500 mg (3.1 to 312 mg/m²), or 2 to 200 mg (1.25 to 125 mg/m²) or 10 to 1000 mg (6.25 to 625 mg/m²), particular examples of doses including 10 mg (6.25 mg/m²), 20 mg (12.5 mg/m²), 50 mg (31.3 mg/m²), 80 mg (50 mg/m²), 100 mg (62.5 mg/m²), 200 mg (125 mg/m²), 300 mg (187.5 mg/m²), 400 mg (250 mg/m²), 500 mg (312.5 mg/m²), 600 mg (375 mg/m²), 700 mg (437.5 mg/m²), 800 mg (500 mg/m²), 900 mg (562.5 mg/m²) and 1000 mg (625 mg/m²).

In some embodiments, a pharmaceutical composition comprising a VEGF variant polypeptide described herein is administered for prophylactic and/or therapeutic treatments. In therapeutic applications, a pharmaceutical composition comprising a VEGF variant polypeptide is administered to an individual already suffering from a disorder, in an amount sufficient to cure or at least partially arrest the symptoms of the disorder. Amounts effective for this use will depend on the severity and course of the disorder, previous therapy, the patient's health status, weight, and response to the drugs, and the judgment of the treating physician.

In prophylactic applications, a pharmaceutical composition comprising a VEGF variant polypeptide described herein is administered to an individual susceptible to or otherwise at risk of a particular disease or disorder. Such an amount is defined to be a “prophylactically effective amount or dose.” In this use, the precise amounts also depend on the patient's state of health, weight, and the like. When used in an individual, effective amounts for this use will depend on the severity and course of the disease, disorder, previous therapy, the patient's health status and response to the drugs, and the judgment of the treating physician.

In some embodiments, a pharmaceutical composition comprising a VEGF variant polypeptide is administered to the patient on a regular basis, e.g., three times a day, two times a day, once a day, every other day or every 3 days. In other embodiments, a pharmaceutical composition comprising a VEGF variant polypeptide is administered to the patient on an intermittent basis, e.g., twice a day followed by once a day followed by three times a day; or the first two days of every week; or the first, second and third day of a week. In some embodiments, intermittent dosing is as effective as regular dosing. In the case wherein the patient's condition does not improve, upon the doctor's discretion the administration of a pharmaceutical composition comprising a VEGF variant polypeptide is administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of a pharmaceutical composition comprising a VEGF variant polypeptide is given continuously; alternatively, the dose of drug being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder is retained. In some embodiments, patients require intermittent treatment on a long-term basis upon any recurrence of symptoms.

The amount of a given agent that will correspond to such an amount will vary depending upon factors such as, disorder and its severity, the identity (e.g., weight) of the subject or host in need of treatment, and is determined according to the particular circumstances surrounding the case, including, for example, the specific pharmaceutical composition comprising a VEGF variant polypeptide being administered, the route of administration, the condition being treated, and the subject or host being treated. The desired dose is conveniently presented in a single dose or as divided doses administered simultaneously (or over a short period of time) or at appropriate intervals, for example as two, three, four or more sub-doses per day.

### Imaging

Disclosed herein, in certain embodiments, are methods for diagnosing an angiogenic disorder in a subject in need thereof comprising: (a) contacting a biological sample from the subject with a labelled hybrid polypeptide of the invention that binds to a biomarker; (b) determining the amount of the biomarker in the biological sample by measuring the amount of the labelled VEGF variant polypeptide bound to the biomarker; (c) comparing the determined amount of the biomarker in the biological sample to an amount of the biomarker in a control; and (d) diagnosing the subject as having an angiogenic disorder based on the comparison.

In some embodiments, the labelling agent comprises a label, a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analog, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, or a combination thereof. In some embodiments, the fluorophore is selected from the group consisting of BODIPY 493/503, BODIPY FL, BODIPY R6G, BODIPY 530/550, BODIPY TMR, BODIPY 558/568, BODIPY 564/570, BODIPY 576/589, BODIPY 581/591, BODIPY TR, Fluorescein, 5(6)-Carboxyfluorescein, 2,7-Dichlorofluorescein, N,N-Bis(2,4,6-trimethylphenyl)-3,4:9,10-perylenebis(dicarboximide, HPTS, Ethyl Eosin, DY-490XL MegaStokes, DY-485XL MegaStokes, Adirondack Green 520, ATTO 465, ATTO 488, ATTO 495, YOYO-1, 5-FAM, BCECF, BCECF, dichlorofluorescein, rhodamine 110, rhodamine 123, Rhodamine Green, YO-PRO-1, SYTOX Green, Sodium Green, SYBR Green I, Alexa Fluor 500, FITC, Fluo-3, Fluo-4, fluoro-emerald, YoYo-1 ssDNA, YoYo-1 dsDNA, YoYo-1, SYTO RNASelect, Diversa Green-FP, Dragon Green, EvaGreen, Surf Green EX, Spectrum Green, Oregon Green 488, NeuroTrace 500525, NBD-X, MitoTracker Green FM, LysoTracker Green DND-26, CBQCA, PA-GFP (post-activation), WEGFP (post-activation), FIASH-CCXXCC, Azami Green monomeric, Azami Green, EGFP (Campbell Tsien 2003), EGFP (Patterson 2001), Fluorescein, Kaede Green, 7-Benzylamino-4-Nitrobenz-2-Oxa-1,3-Diazole, Bex1, Doxorubicin, Lumio Green, IRDye 800, IRDye 750, IRDye 700, DyLight 680, DyLight 755, DyLight 800 and SuperGlo GFP. In some embodiments, the labelling agent is selected from the group consisting of: a positron-emitting isotope (such as ¹⁸F), a gamma-ray isotope (such as ^{99m}Tc), a paramagnetic molecule or nanoparticle (such as a coated magnetite nanoparticle), a gadolinium chelate (such as diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA)), an iron oxide particle, a super paramagnetic iron oxide particle, an ultra small paramagnetic particle, a manganese chelate, a gallium containing agent, a technetium chelate (such as HYNIC, DTPA, and DOTA), a copper chelate, a radioactive fluorine, a radioactive iodine, a indium chelate, or a radioactive moiety (such as ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu radioactive isotopes of Lu). In some embodiments, the connecting moiety connects the labelling agent to the VEGF variant polypeptide. In some embodiments, the connecting moiety is selected from the group consisting of a bond, a peptide, a polymer, a water soluble polymer, optionally substituted alkyl, optionally substituted heteroalkyl, optionally substituted heterocycloalkyl, optionally substituted cycloalkyl, optionally substituted heterocycloalkylalkyl, optionally substituted heterocycloalkylalkenyl, optionally substituted aryl, optionally substituted heteroaryl, and optionally substituted heterocycloalkylalkenylalkyl. In some embodiments, the connecting moiety is 4′-phosphopantetheine.

In some embodiments, the fluorophore is selected from the group consisting of: BODIPY 493/503, BODIPY FL, BODIPY R6G, BODIPY 530/550, BODIPY TMR, BODIPY 558/568, BODIPY 564/570, BODIPY 576/589, BODIPY 581/591, and BODIPY TR. In some embodiments, the fluorophore is BODIPY FL. In some embodiments, the fluorophore is not BODIPY 530. In some embodiments, the fluorophore has an excitation maxima of between about 500 and about 600 nm. In some embodiments, the fluorophore has an excitation maxima of between about 500 and about 550 nm. In some embodiments, the fluorophore has an excitation maxima of between about 550 and about 600 nm. In some embodiments, the fluorophore has an excitation maxima of between about 525 and about 575 nm. In some embodiments, the fluorophore has an emission maxima of between about 510 and about 670 nm. In some embodiments, the fluorophore has an emission maxima of between about 510 and about 600 nm. In some embodiments, the fluorophore has an emission maxima of between about 600 and about 670 nm. In some embodiments, the fluorophore has an emission maxima of between about 575 and about 625 nm.

In some embodiments, the fluorophore is fluorescein or indocyanine green.

In some embodiments, the fluorophore is ATTO 488, DY-547 or DY-747.

In some embodiments, the labelling agent is a positron-emitting isotope (e.g., ¹⁸F) for positron emission tomography (PET), gamma-ray isotope (e.g., ^{99m}Tc) for single photon emission computed tomography (SPECT), or a paramagnetic molecule or nanoparticle (e.g., Gd³⁺ chelate or coated magnetite nanoparticle) for magnetic resonance imaging (MRI).

In some embodiments, the labelling agent is: a gadolinium chelate, an iron oxide particle, a super paramagnetic iron oxide particle, an ultra small paramagnetic particle, a manganese chelate or gallium containing agent. Examples of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA).

In some embodiments, the labelling agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, the labelling agent is a nuclear probe. In some embodiments, the imaging agent is a SPECT or PET radionuclide probe. In some embodiments, the radionuclide probe is selected from: a technetium chelate, a copper chelate, a radioactive fluorine, a radioactive iodine, a indium chelate. Examples of Tc chelates include, but are not limited to HYNIC, DTPA, and DOTA.

In some embodiments, the labelling agent is a radioactive moiety, for example a radioactive isotope such as ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu radioactive isotopes of Lu, and others.

In some embodiments, the polypeptide of the invention further comprises a Sfp tag that is at least 90%, at least 95%, at least 99%, or 100% identical to a peptide sequence of DSLEFIASKLA.

In some embodiments, a labelled hybrid polypeptide of the invention comprises the hybrid polypeptide, a connecting moiety, and a labelling agent. In some embodiments, the connecting moiety connects the labelling agent to the polypeptide. In some embodiments, the connecting moiety is selected from a bond, a peptide, a polymer, a water soluble polymer, optionally substituted alkyl, optionally substituted heteroalkyl, optionally substituted heterocycloalkyl, optionally substituted cycloalkyl, optionally substituted heterocycloalkylalkyl, optionally substituted heterocycloalkylalkenyl, optionally substituted aryl, optionally substituted heteroaryl, and optionally substituted heterocycloalkylalkenylalkyl. In some embodiments, the connecting moiety is an optionally substituted heterocycle. In some embodiments, the heterocycle is selected from aziridine, oxirane, episulfide, azetidine, oxetane, pyrroline, tetrahydrofuran, tetrahydrothiophene, pyrrolidine, pyrazole, pyrrole, imidazole, triazole, tetrazole, oxazole, isoxazole, oxirene, thiazole, isothiazole, dithiolane, furan, thiophene, piperidine, tetrahydropyran, thiane, pyridine, pyran, thiapyrane, pyridazine, pyrimidine, pyrazine, piperazine, oxazine, thiazine, dithiane, and dioxane. In some embodiments, the heterocycle is piperazine. In further embodiments, the connecting moiety is optionally substituted with a halogen, CN, OH, NO₂, alkyl, S(O), and S(O)₂. In other embodiments, the water soluble polymer is a PEG group.

In some embodiments, the angiogenic disorder is ocular neovascularization, choroidal neovascularization, iris neovascularization, corneal neovascularization, retinal neovascularization, pterygium, pannus, pinguecula, diabetic retinopathy, diabetic macular edema, retinal detachment, posterior uveitis, macular degeneration, a keloid, glaucoma, cataract, partial blindness, complete blindness, myopia, myopic degeneration, deterioration of central vision, metamophospsia, color disturbances, hemorrhaging of blood vessels, or retinal vein occlusion.

In some embodiments, the angiogenic disorder is a cancer. In some embodiments, the cancer is prostate cancer, breast cancer, lung cancer, esophageal cancer, colon cancer, rectal cancer, liver cancer, urinary tract cancer (e.g., bladder cancer), kidney cancer, lung cancer (e.g., non-small cell lung cancer), ovarian cancer, cervical cancer, endometrial cancer, pancreatic cancer, stomach cancer, thyroid cancer, skin cancer (e.g., melanoma), hematopoietic cancers of lymphoid or myeloid lineage, head and neck cancer, nasopharyngeal carcinoma (NPC), glioblastoma, teratocarcinoma, neuroblastoma, adenocarcinoma, cancers of mesenchymal origin such as a fibrosarcoma or rhabdomyosarcoma, soft tissue sarcoma and carcinoma, choriocarcinioma, hepatoblastoma, Karposi's sarcoma or Wilm's tumor.

In some embodiments, the angiogenic disorder is an inflammtory disorder. In some embodiments, the inflammatory disorder is inflammatory arthritis, osteoarthritis, psoriasis, chronic inflammation, irritable bowel disease, lung inflammation or asthma. In some embodiments, the angiogenic disorder is an autoimmune disorder. In some embodiments, the autoimmune disorder is rheumatoid arthritis, multiple sclerosis, or systemic lupus erythematosus.

In some embodiments, the biomarker is a biomarker of an angiogenic disorder. In some embodiments, the growth factor receptor is a vascular endothelial growth factor receptor (VEGFR). In some embodiments, the VEGFR is VEGFR1 or VEGFR2. In some embodiments the growth factor receptor is PDGFR-α or PDGFR-β.

In some embodiments, the biomarker is a combination of biomarkers. In some embodiments, the combination of biomarkers comprises VEGFR1, VEGFR2, PDGFR-a and PDGFR-β. In some embodiments, the measuring the amount of the labelled hybrid polypeptide bound to the biomarker comprises a detection method. In some embodiments, the detection method is selected from the group consisting of Western Blot, immunoprecipitation, enzyme-linked immunosorbent assay (ELISA), immunohistochemistry, and radioimmunoassay. In some embodiments, the detection method is selected from the group consisting of spectroscopic, photochemical, biochemical, radiographical, immunochemical, chemical, electrical, and optical detection methods. In some embodiments, the detection method comprises detecting the concentration or the presence of the labelling agent. In some embodiments, the biological sample comprises tissue. In some embodiments, the biological sample comprises pterygium tissue. In some embodiments, the biological sample is in vivo or ex vivo.

The invention also provides methods for assessing a response of a subject to a therapy for treatment of an angiogenic disorder comprising: (a) contacting a first biological sample from the subject with a labelled hybrid polypeptide of the invention that binds to a biomarker and determining the amount of the biomarker in the first biological sample by measuring the amount of the labelled polypeptide bound to the biomarker; (b) contacting a second biological sample from the subject with the labelled polypeptide after the subject has been administered a therapeutic agent and determining the amount of the biomarker in the second biological sample by measuring the amount of the labelled polypeptide bound to the biomarker; and (c) determining whether the subject has a positive, negative, or neutral response to the therapy based on a comparison of the amounts of the biomarker in the first and second biological samples.

In some embodiments, the amount of the biomarker in a first biological sample is determined before treatment with a therapeutic agent, for example a therapeutic hybrid polypeptide of the invention. In some embodiments, the amount of the biomarker in a second biological sample is determined after completion of a treatment regimen with the therapeutic agent, for example 1 week, 2 weeks, 1 month, 2 months, or 6 months after completion of treatment regimen.

In some embodiments, determining the amount of biomarker in a sample or control comprises in vivo imaging, non-invasive or invasive. In some embodiments, determining the amount of biomarker in a sample or control comprises ex vivo imaging. In some embodiments, the biological sample is a biopsy sample or an aspiration sample.

The selection of a diagnostic control depends on the type of control (positive or negative), the type of biological sample, and whether the imaging is in vivo or ex vivo. For example, where the biological sample is an eye (for in vivo screening of an angiogenesis-related ocular disorder), in some embodiments, the negative control is the subject's healthy, non-affected eye. In some embodiments, the negative control is the average concentration of the biomarker present in a population of healthy, un-related, eyes where it is known that the subject does not suffer from any disease or condition that involves angiogenesis. For ex vivo determination of the biomarker concentration, such as ex vivo determination of the amount of the biomarker in a biopsy sample, is some embodiments, the control is a biopsy sample taken at an early date. In some embodiments, the control is subjected to the treatment as the biological sample.

In some embodiments the diagnostic absence, diagnostic presence, or change in the amount of a biomarker of an angiogenic disorder, for example an angiogenesis-associated disorder, is predictive of whether a therapy will be effective, or whether a therapy is having an effect. The individual may be treated with a hybrid polypeptide of the invention in accordance with the diagnosis.

### Kits

Disclosed herein, in certain embodiments, are kits comprising a VEGF variant polypeptide or Fc-VEGF variant polypeptides.

The kits, regardless of type, will generally include one or more containers into which the biological agents are placed and, preferably, suitably aliquoted. In some embodiments, the components of the kits are packaged either in aqueous media or in lyophilized form.

In a further embodiment, the present invention provides kits containing a VEGF variant polypeptide or Fc-VEGF variant polypeptide, which are used, for instance, for therapeutic or non-therapeutic applications. The kit comprises a container with a label. Suitable containers include, for example, bottles, vials, and test tubes. In some embodiments, the containers are formed from a variety of materials such as glass or plastic. The container holds a composition which includes a VEGF variant polypeptide or Fc-VEGF variant polypeptide that is effective for therapeutic or non-therapeutic applications, such as described above. The label on the container indicates that the composition is used for a specific therapy or non-therapeutic application, and also indicates directions for either in vivo or in vitro use, such as those described above.

The kit will typically comprise the container described above and one or more other containers comprising materials desirable from a commercial and user standpoint, including buffers, diluents, filters, needles, syringes, and package inserts with instructions for use. In some embodiments, the kit also includes a control consisting of wild-type VEGF.

### EXAMPLES

### Example 1—Generation of Single-Chain VEGF Variant

A single-chain variant of VEGF (termed scVEGF), in which two monomeric VEGF chains were physically tethered through a flexible linker, was created. Point mutations were introduced into scVEGF (chain 1: F17A, E64G; chain 2: I46A, I83A) to generate scVEGF_{MUT} that conferred antagonistic activity on this variant by blocking a second molecule of VEGFR2 from binding to this pole. Once single-chain VEGF variants were established, a 9-11 amino-acid integrin binding loop was introduced into scVEGF in place of residues 83-89 (i.e. loop 3), which is on the same pole as the point mutations listed above to (potentially) allow binding to integrin receptor instead of VEGFR2 at this pole.

### Example 2—Engineering Optimal Linkers of Single Chain VEGF Antagonists

The linker moiety, which connects the C-terminus of monomer A to the N-terminus of monomer B, was optimized on the scVEGF-mE7I (SEQ ID No.: 75) construct to improve protein expression yield and binding affinity to endothelial cells. Three linkers of varying lengths and compositions were designed, and are shown in Table 2 along with the original linker sequence. The linkers shown in Table 2 utilize glycine and serine residues which are not expected to form any secondary structures, and are also known to have lower immunogenicity.

TABLE 2
Exemplary Linker Sequences

| Linker | Amino Acid | |
|---|---|---|
| Construct | Length | Sequence |
| Original Linker | 14 | GSTSGSGKSSEGKG |
| | | (SEQ ID NO: 41) |
| L1A | 19 | GSTSGSGKSSEGKGGGGGS |
| | | (SEQ ID NO: 42) |
| L2A | 14 | GGGGSGGGGSGGGG |
| | | (SEQ ID NO: 43) |
| L3A | 20 | GGGGSGGGGSGGGGSGGGGS |
| | | (SEQ ID NO: 44) |

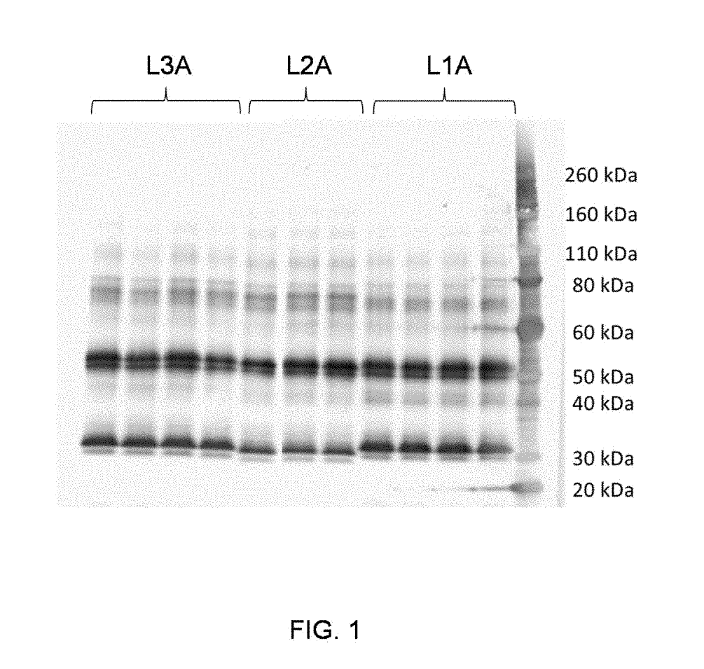

Small scale expression of constructs containing either L1A, L2A, or L3A were produced. As shown in FIG. 1, longer linkers L1A and L3A afforded higher yield of the desired protein (shown at approximately 30 kDa). Because L3A contained only glycine and serine residues and therefore had lower potential for immunogencity (as compared to L1A) it was selected as the optimized linker. The total improvement in final yield with L3A compared to the construct with the original linker was ˜2-3 fold.

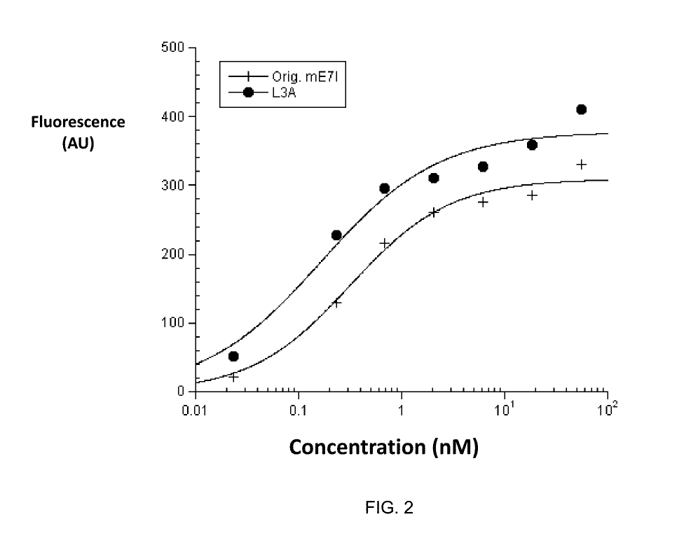

The cell binding assay on human endothelial cells was performed to compare target binding affinity of a construct containing L3A to a construct with the original linker (scVEGFmE7I, SEQ ID: 75). As shown in FIG. 2, the scVEGF-mE construct containing the original linker had a K_{D} of 0.32±0.07 nM, while the scVEGF-mE construct containing the L3A linker had a K_{D} of 0.16± of 0.06 nM representing an ˜2-fold improvement.

### Example 3—Identifying the Minimal Set of Mutations for High-Affinity Binding

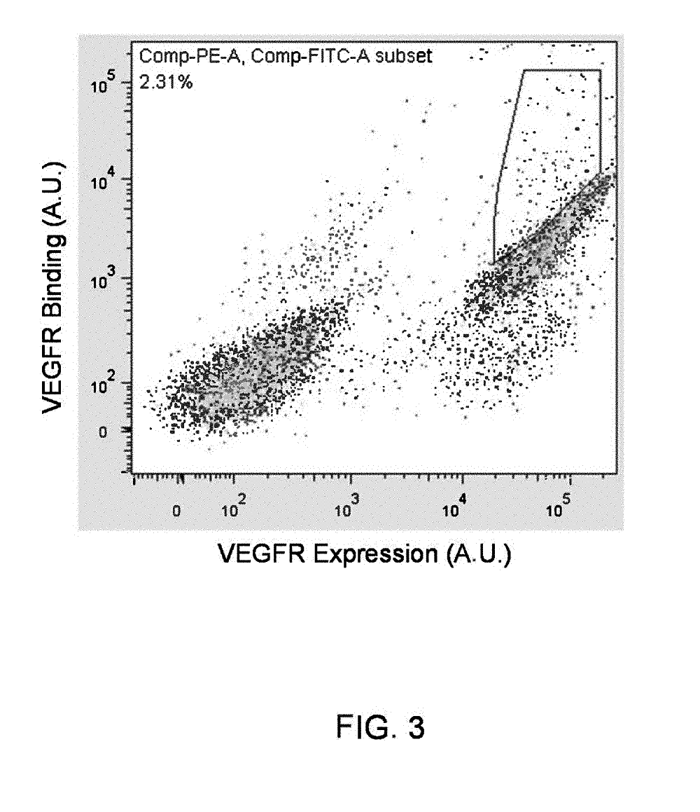

The scVEGFmutE construct contains 7 mutations; Chain 1 contains mutations at F36L, E44G, D63G, and Q87R, and Chain 2 contains mutations at K16R, D41N, and D63N. To identify the minimal set of mutations is required for high-affinity binding, a library of 2⁷ mutants was generated (where the residue in each of the 7 positions was independently allowed to be either the residue found in scVEGFmutE or the wild-type scVEGF) and tested for VEGFR2 binding on yeast using an appropriate amount of soluble VEGFR2-Fc as a probe. Analysis of the population of yeast that retained high affinity binding to VEGFR2 (FIG. 3) showed that the mutations Chain 1 F36L, Chain 1E44G, Chain 1Q87R, and Chain 2 D63N are enriched, while Chain 1 D63G, Chain 2 K16R, and Chain 2 D41N are not. Chain 1 E44G and Chain 2 D63N were universally enriched, while Chain 1 F36L and Chain 1 Q87R were strongly enriched. These results imply that the following set of mutations: Chain 1 F36L, Chain 1E44G, Chain 1Q87R, and Chain 2 D63N are necessary and sufficient to confer high-affinity VEGFR2 binding.

### Example 4—Fc-Fusions of scVEGF Constructs

scVEGF constructs were modified with Fc fusions in order to 1) increase size beyond renal cutoff which improves circulation of half-life with systemic administration and thereby allowing less frequent dosing of therapeutics, and 2) leverage the immune system complement and effector functions for more potent activity. scVEGF-Fc fusions were examined for retained binding affinity as in the parent scVEGF, and for retained antagonistic activity of the parent scVEGF.

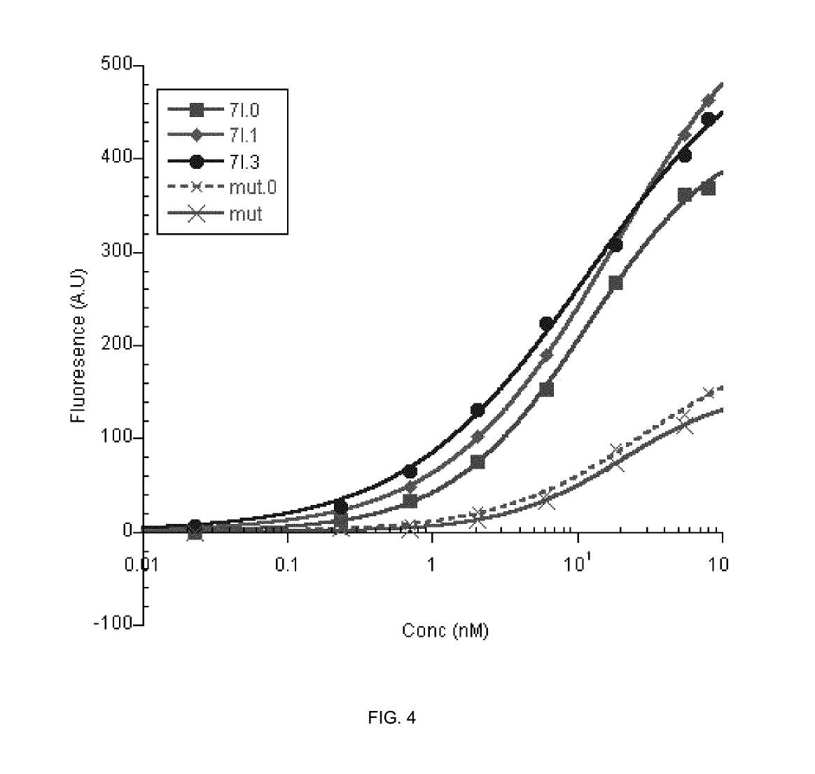

First, scVEGF constructs were evaluated in a cell-binding assay on human endothelial cells (HUVECs). As shown in FIG. 4, the binding affinity of scVEGF-Fc fusion is unchanged compared to the parent scVEGF (compare mut.0 curve and mut curve). Further, because scVEGF binds two different cell-surface receptors (VEGFR and integrin), the corresponding Fc-fusion which is dimeric will bind a total of four receptors. To test if the resultant steric crowding impacts binding, varying lengths of Gly₄Ser linker at the fusion junction of scVEGF and the Fc domain were tested. Three different linker lengths with 0, 1, or 3 Gly₄Ser repeats (as shown as 71.0 vs. 71.1 vs. 71.3 in FIG. 4) were tested, and no significant differences were observed. Therefore, in some embodiments, scVEGF is directly fused to the Fc region.

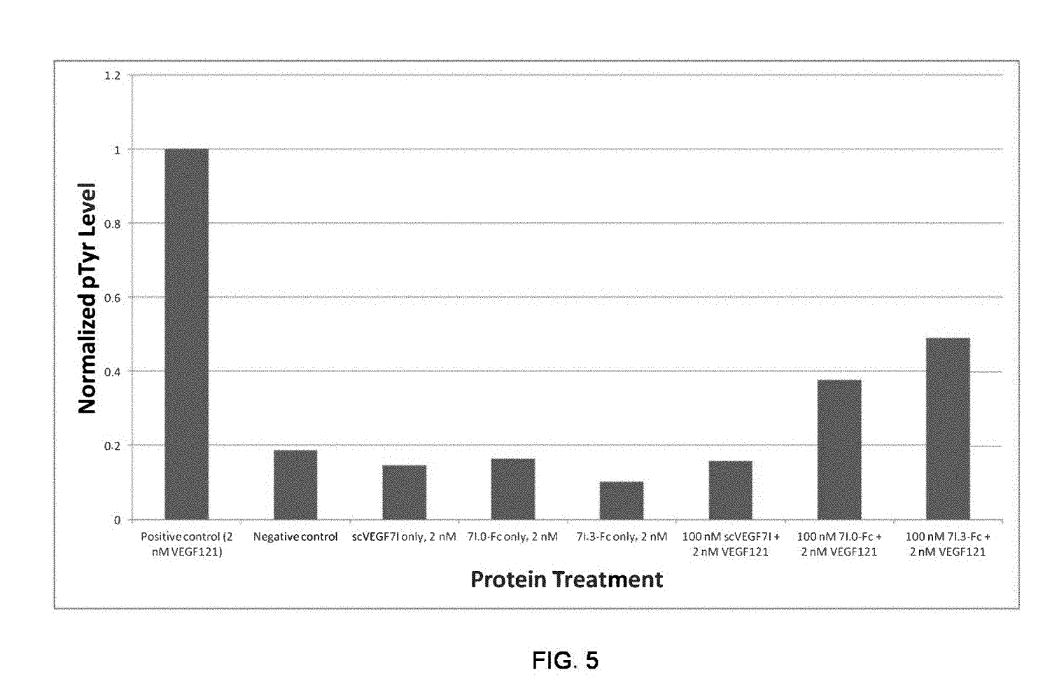

Next, scVEGF-Fc fusion constructs were evaluated for antagonistic activity in a phosphorylation assay on HUVECs (FIG. 5). Columns 4 and 5 when compared to the positive and negative controls (columns 1 and 2, respectively) demonstrate that the Fc-fusions are not agonists. The comparison of columns 6 and 7 with positive and negative controls (columns 1 and 2, respectively) demonstrated that the Fc-fusiona retain antagonistic activity similar to the scVEGF equivalent (compare columns 6 and 7 to column 5).

### Example 5—Characterization of scVEGF Constructs Binding to VEGFR1

The binding and antagonistic properties of the parent construct scVEGFMUT (mut) was compared to that of the affinity-matured variant scVEGFMUT-E (mE, SEQ ID NO.: 55). As shown below in Table 3, a 12-fold change in R1/R2 selectivity was observed going from mut to mE. Nonetheless, the affinity-matured variant scVEGFMUT-E retained binding to VEGFR1 with an affinity of 550 pM.

TABLE 3
Comparison of scVEGFmut and scVEGFmE Constructs

| | VEGFR1 (nM) | VEGFR2 (nM) | | |
|---|---|---|---|---|
| | (on PAE- | (on PAE- | Selectivity | HUVEC |
| Protein | VEGFR1 cells) | VEGFR2 cells) | (R2 over R1) | (nM) |
| mut | 4.5 | 50 | 0.09 | 44 |
| mE | 0.55 | 0.5 | 1.1 | 0.4 |

### Example 2—Treatment of bFGF-Neovascularization with scVEGF

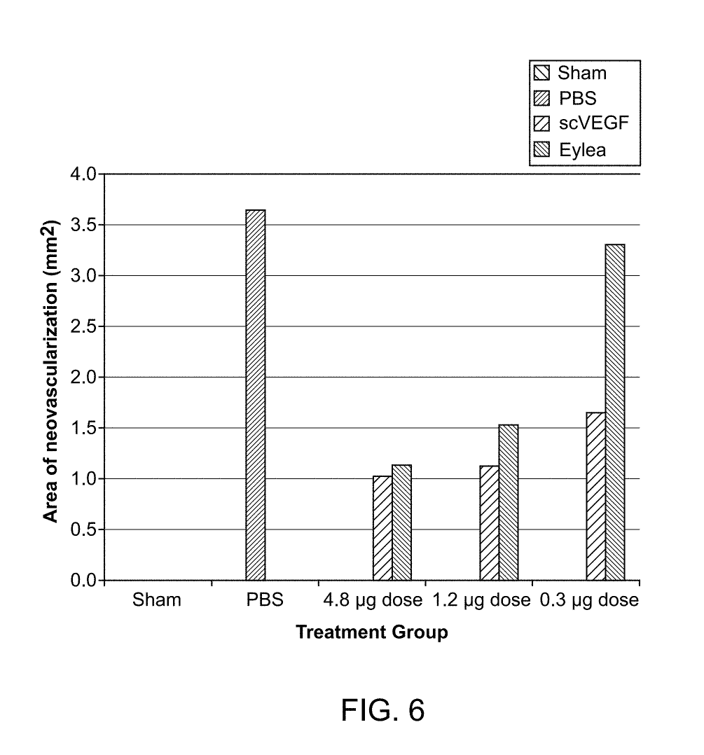

The bFGF-induced corneal neovascularization model was performed as previously described by Kenyon et. al (1996) Invest. Ophthalmol. Vis. Sci. 37:1625 with suitable modifications, including, using 100 ng of bFGF/pellet, formulating the pellet with the agent to be tested, and measuring extent of neovascularization on day 6 post-pellet implantation. The results are presented in FIG. 6. The scVEGF variant polypeptide of SEQ ID No:75 was able to inhibit neovascularization at all doses tested. Notably, it was either as potent as, or more potent than, a clinically approved angiogenesis inhibitor, at all doses tested. Furthermore, the scVEGF variant polypeptide was also more potent that the corresponding variant (SEQ ID No.: 78) in which VEGFR1 binding was eliminated through the introduction of mutations that are known to simultaneously retain VEGFR2 binding.

### Example 3

### Identification of Biomarkers for Anti-Angiogenic Therapeutic Intervention in Ocular Diseases

Tissue specimens obtained from consenting patients undergoing clinically indicated pterygium removal surgery were subsequently tested for markers of angiogenic vasculature.

Tissues were fixed in formalin before paraffin processing, embedding, and were sectioned at 5 μm onto Superfrost Plus slides. Pterygium tissue sections were deparaffinized in xylene and rehydrated through a graded alcohol series to water. The slides were subjected to heat-mediated antigen retrieval in sodium citrate buffer. Slides were washed 3×5 min in PBS, then incubated in 10% normal goat serum in PBS with 1% BSA for 3 hrs at RT for blocking. Each section was then incubated for 12 hrs at 4 C with a cocktail of two antibodies raised in differing species to achieve staining overlays. Antibodies for von Willebrand Factor (vWF), CD31, VEGFR1, VEGFR2, β3 integrin (to probe for αᵥβ₃ integrin), β5 integrin (to probe for αᵥβ₅ integrin), α5 integrin (to probe for α_{c}β₁ integrin), pro-MMP2, and MMP2 were used. PBS with 1% BSA was used for all antibody dilutions. The slides were then washed 3×5 min in PBS, and incubated for 1 hr at RT in Alexa Fluor 488 and Alexa Fluor 594 conjugated antibodies raised in goat against mouse and rabbit, respectively. Slides were then washed 3×5 min in PBS, and mounted with 4′-6-diamidino-2-phenylindole (DAPI)-containing Vectashield mounting media.

Fluorescence images were captured using a 10× Plan Apochromat objective on an Axiolmager Z1 Epifluorescence Microscope with appropriate filter sets. Exposure times for each antigen were constant across samples. All images of an antigen received the same linear brightness and contrast adjustments using Zen Blue software.

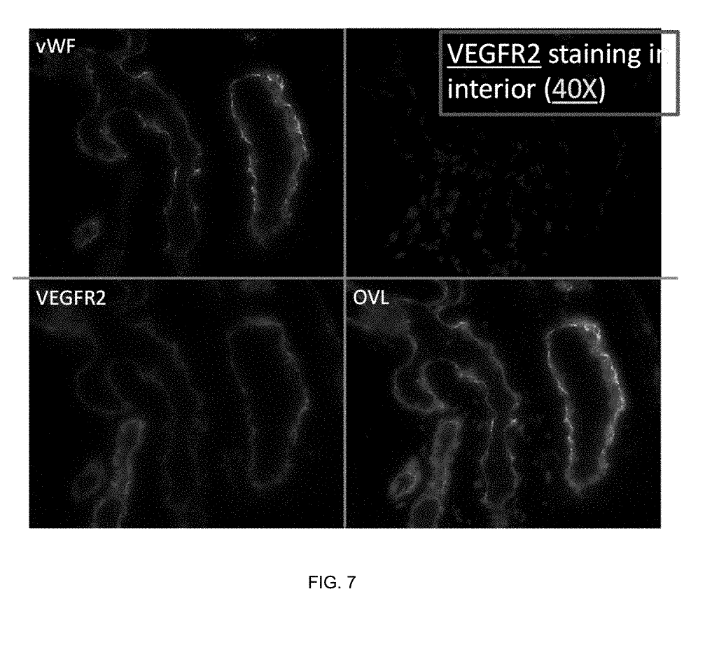

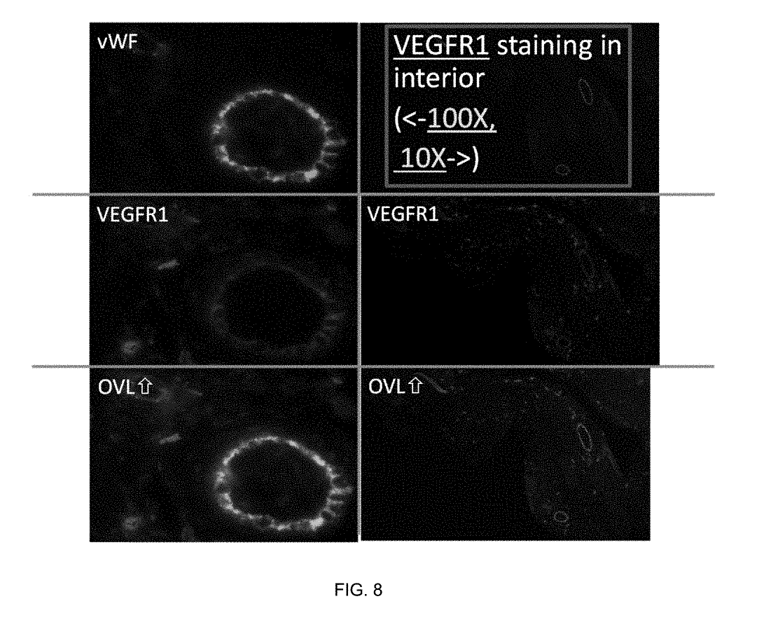

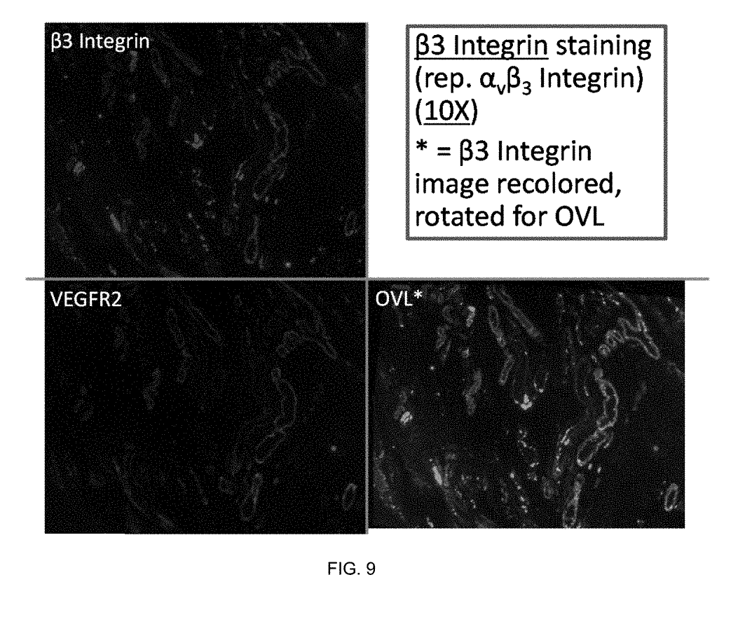

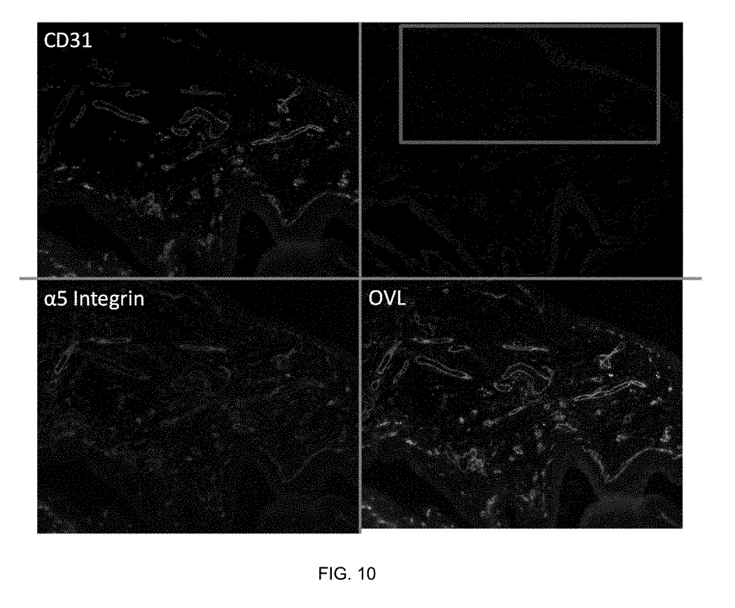

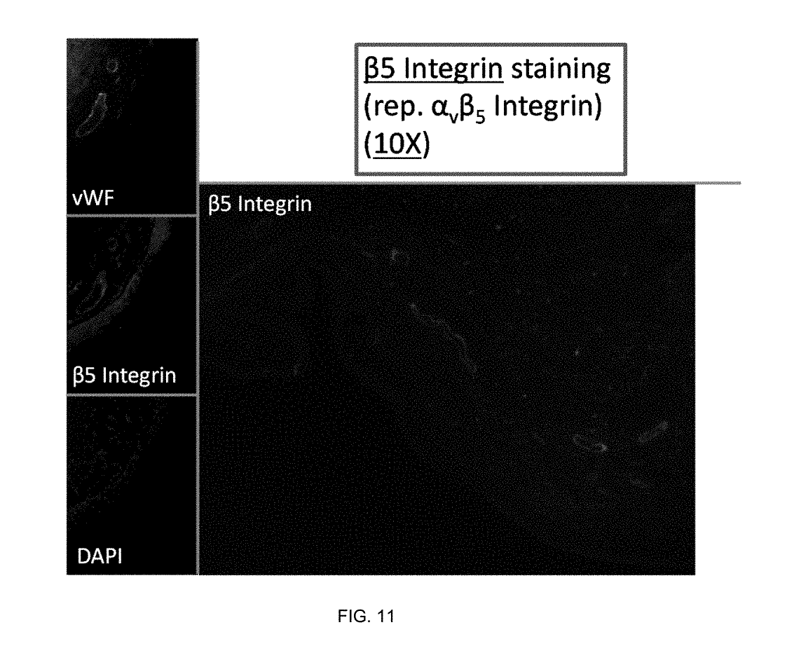

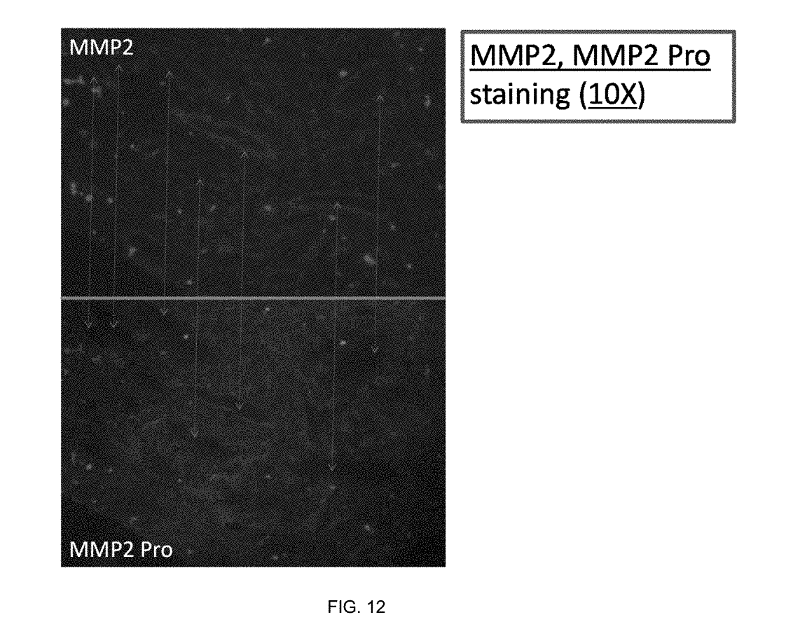

As shown in FIGS. 7-12 are fluorescence images. FIG. 7 exemplifies immunohistochemical staining of von Willebrand Factor (vWF) and VEGFR2 in human pterygium. FIG. 8 exemplifies immunohistochemical staining of vWF and VEGFR1 in human pterygium. FIG. 9 exemplifies immunohistochemical staining of αᵥβ₃ integrin and VEGFR2 in human pterygium. FIG. 10 exemplifies immunohistochemical staining of CD31 and α₅β₁ integrin in human pterygium. FIG. 11 exemplifies immunohistochemical staining of CD31, and αᵥβ₅ integrin in human pterygium. FIG. 12 exemplifies immunohistochemical staining of MMP2, pro-MMP2, and CD31 in human pterygium. In all cases, prominent staining with all markers that were tested was observed. Significantly, in all cases this staining co-localized with known markers of endothelial cells (vWF or CD31) confirming that the expression of these markers are associated with endothelial cells. Furthermore, by using antibodies with complimentary specificities for MMP2 we were able to show that only the active form of MMP2 co-localizes with marker for endothelial cells. In particular, an antibody that can detect both active MMP2 and pro-MMP2 showed prominent vascular staining (FIG. 7 top-left panel). In contrast, an antibody that exclusively recognizes the pro-MMP2 form did not show any visible staining for corresponding vessels (FIG. 7 top-right panel).

### Example 4—Clinical Trial Using a VEGF Variant Polypeptide with Pterygium

The purpose of this study is to investigate whether a VEGF variant polypeptide disclosed herein can halt or cause regression of a pterygium growth. The VEGF variant polypeptide is applied topically directly onto the pterygium growth once a day for six months.

Study Type: Interventional

Study Design: Interventional Model: Single Group Assignment

Masking: Open Label

Primary Purpose: Treatment

Primary Outcome Measures: The area of the pterygium enlarged or regressed as measured from the limbus before and after VEGF variant polypeptide administration; Time frame: baseline and 3 months

Growth of the pterygium is defined as an increase in the area of the pterygium as is measured from the limbus toward the visual axis.

Regression of the pterygium is defined as a decrease in the pterygium length as is measured from the limbus toward the visual axis.

Secondary Outcome Measures: Number of patients having surgical removal of pterygium; Time Frame: 12 months

Eligibility: Ages Eligible for Study: 19 years and older; Genders Eligible for Study: Both; Accepts Healthy Volunteers: No

Inclusion Criteria: 19 years of age and older; Diagnosis of pterygium; Healthy enough to make scheduled follow-up visits

Exclusion Criteria: Women of childbearing potential and males who plan to father a child during their participation in the study will be excluded from the study.

1. A VEGF variant polypeptide, comprising the formula:
A-L-B,
wherein
A is a first VEGF monomer subunit;
B is a second VEGF monomer subunit; and
L is a peptide linker having a formula selected from: (GS)ₙ, wherein n is an integer from 6 to 15; (G₂S)ₙ, wherein n is an integer from 4 to 10; (G₃S)ₙ, wherein n is an integer from 3 to 8; (G₄S)ₙ, wherein n is an integer from 2 to 6; (G)ₙ, wherein n is an integer from 12 to 30; and (S)ₙ, wherein n is an integer from 12 to 30.

2. A VEGF variant polypeptide of claim 1, comprising the formula:
A-L₁-B-(L₂-A-L₁-B)ₙ-L₂-A-L₁-B,
wherein
A is a first VEGF monomer subunit,
B is a second VEGF monomer subunit,
L₁ is a peptide linker having 14 to 20 amino acids;
L₂ is a peptide linker; and
n is an integer from 0 to 4.

3. The VEGF variant polypeptide of claim 2, wherein L₁ is a peptide linker having a formula selected from: (GS)ₙ, wherein n is an integer from 6 to 15; (G₂S)ₙ, wherein n is an integer from 4 to 10; (G₃S)ₙ, wherein n is an integer from 3 to 8; (G₄S)ₙ, wherein n is an integer from 2 to 6; (G)ₙ, wherein n is an integer from 12 to 30; and (S)ₙ, wherein n is an integer from 12 to 30.

4. The VEGF variant polypeptide of claim 1, wherein L or L₁ is selected from the group consisting of: GSTSGSGKSSEGKGGGGGS (SEQ ID NO: 42); GGGGSGGGGSGGGG (SEQ ID NO: 43); and GGGGSGGGGSGGGGSGGGGS (SEQ ID NO: 44).

5. The VEGF variant polypeptide of claim 2, wherein L₂ is selected from the group consisting of: (GS)ₙ, where n=10-30; (G₂S)ₙ, where n=6-20; (G₃S)ₙ, where n=5-15; (G₄S)ₙ, where n=4-12; (G)ₙ, where n=20-60; and (S)ₙ, where n=20-60.

6. (canceled)

7. The VEGF variant polypeptide of claim 1,
wherein the VEGF variant polypeptide is a bifunctional antagonist that antagonizes VEGFR1 or VEGFR2, an αᵥβ₃, αᵥβ₅ or α₅β₁ integrin, or combination thereof.

8-9. (canceled)

10. The VEGF variant polypeptide of claim 1, wherein at least one of the VEGF monomer subunits is a VEGF-A monomer selected from VEGF₁₆₅; VEGF_{165b}; VEGF₁₂₁; VEGF₁₄₅; VEGF₁₈₉; VEGF₂₀₆.

11. (canceled)

12. The VEGF variant polypeptide of claim 1, wherein at least one of the VEGF monomer subunits is a VEGF-B subunit; a VEGF-C subunit; a VEGF-D subunit; a PIGF.

13. (canceled)

14. The VEGF variant polypeptide of claim 1, wherein one VEGF monomer subunit comprises at least one mutation selected from the group consisting of: V14A, V14I, V15A, K16R, F17L, M18R, D19G, Q22R, R23K, I29V, L32S, I35V, F36L, F36S, D41N, E42K, E44G, Y45H, F47S, K48E, P49L, S50P, P53S, G58S, C60Y, D63H, D63N, D63G, I76T, M78V, M81T, M81V, R82G, H86Y, Q87R, Q89H, H90R, I91T, I91V, N100D, and K101E.

15. The VEGF variant polypeptide of claim 14, wherein one VEGF monomer subunit comprises at least one mutation selected from the group consisting of F36L, E44G, D63G, and Q87R.

16. The VEGF variant polypeptide of claim 14, wherein one VEGF monomer subunit comprises at least one mutation selected from the group consisting of F36L, E44G, and Q87R.

17. The VEGF variant polypeptide of claim 14, wherein one VEGF monomer subunit comprises at least one mutation selected from the group consisting of V14A, V14I, V15A, K16R, F17L, M18R, D19G, Q22R, R23K, I29V, L32S, I35V, F36L, F36S, D41N, E42K, E44G, Y45H, F47S, K48E, P49L, S50P, P53S, G58S, C60Y, D63H, D63N, D63G, I76T, M78V, M81T, M81V, R82G, H86Y, Q87R, Q89H, H90R, I91T, I91V, N100D, and K101E.

18. The VEGF variant polypeptide of claim 14, wherein the VEGF monomer subunit comprises at least one mutation selected from the group consisting of K16R, D41N, and D63N.

19. (canceled)

20. The VEGF variant polypeptide of claim 1, wherein the first or the second or both of the VEGF monomer subunits comprises an RGD loop.

21. The VEGF variant polypeptide of claim 20, wherein the RGD loop is at least 90%, at least 95%, at least 99%, or 100% identical to a sequence selected from the group consisting of SEQ ID NOs: 1-40, 66-72.

22. The VEGF variant polypeptide of claim 20, wherein the RGD containing loop replaces loop 1, loop 2, or loop 3 of the first or the second VEGF monomer subunit, or any combinations thereof.

23. The VEGF variant polypeptide of claim 1, wherein the VEGF variant polypeptide is at least 90%, at least 95%, at least 99%, or 100% identical to a sequence of mE7I (SEQ ID NO: 75); mA7I (SEQ ID NO: 76); mJ7I (SEQ ID NO: 77); or mE7I-R1null (SEQ ID NO: 78).

24. The VEGF variant polypeptide of claim 1, wherein the VEGF variant polypeptide further comprises a toxin.

25-28. (canceled)

29. An Fc-VEGF variant polypeptide according to claim 1, wherein the variant polypeptide is fused to an immunoglobulin Fc region.

30. A method of treating an angiogenic disorder in an individual in need thereof, comprising administering to the individual an Fc-VEGF variant polypeptide fusion according to claim 29.

31. The method of claim 30, wherein the angiogenic disorder is pterygium.

32. (canceled)

33. The method of claim 30, wherein the angiogenic disorder is a cancer.

34. (canceled)

35. The method of claim 30, wherein the angiogenic disorder is an inflammatory disorder.

36. (canceled)

37. The method of claim 30, wherein the angiogenic disorder is an autoimmune disorder.

38-39. (canceled)

40. A method of non-surgically treating or preventing recurrence of a disorder characterized by neovascularization of the external surface of an eye, including the cornea and bulbar conjunctiva, of a subject in need thereof, comprising administering to the subject an effective amount of a pharmaceutical composition comprising a composition of claim 1.

41. (canceled)

42. The method of claim 40, wherein the pharmaceutical composition is formulated as an ophthalmically acceptable solution, gel, cream or ointment.

43. The method of claim 40, wherein the disorder characterized by neovascularization of the external surface of the eye is pterygium.

44-45. (canceled)


SEQUENCE LISTING (78 sequences)

SEQ ID NO: 1 - protein, 9 aa, Artificial sequence
  Synthetic polypeptide
PFGTRGDSS

SEQ ID NO: 2 - protein, 9 aa, Artificial sequence
  Synthetic polypeptide
SGERGDGPT

SEQ ID NO: 3 - protein, 9 aa, Artificial sequence
  Synthetic polypeptide
SDGRGDGSV

SEQ ID NO: 4 - protein, 9 aa, Artificial sequence
  Synthetic polypeptide
PIGRGDGST

SEQ ID NO: 5 - protein, 9 aa, Artificial sequence
  Synthetic polypeptide
LAERGDSSS

SEQ ID NO: 6 - protein, 9 aa, Artificial sequence
  Synthetic polypeptide
PTGRGDLGA

SEQ ID NO: 7 - protein, 9 aa, Artificial sequence
  Synthetic polypeptide
RGIRGDSGA

SEQ ID NO: 8 - protein, 9 aa, Artificial sequence
  Synthetic polypeptide
VGGRGDVGV

SEQ ID NO: 9 - protein, 9 aa, Artificial sequence
  Synthetic polypeptide
ITARGDSFG

SEQ ID NO: 10 - protein, 9 aa, Artificial sequence
  Synthetic polypeptide
ITERGDSGH

SEQ ID NO: 11 - protein, 9 aa, Artificial sequence
  Synthetic polypeptide
PQARGDRSD

SEQ ID NO: 12 - protein, 9 aa, Artificial sequence
  Synthetic polypeptide
SRTRGDASD

SEQ ID NO: 13 - protein, 9 aa, Artificial sequence
  Synthetic polypeptide
PAARGDGGL

SEQ ID NO: 14 - protein, 9 aa, Artificial sequence
  Synthetic polypeptide
PVARGDSGA

SEQ ID NO: 15 - protein, 9 aa, Artificial sequence
  Synthetic polypeptide
PQQRGDGPH

SEQ ID NO: 16 - protein, 9 aa, Artificial sequence
  Synthetic polypeptide
PLPRGDGQR

SEQ ID NO: 17 - protein, 9 aa, Artificial sequence
  Synthetic polypeptide
HAGRGDSPS

SEQ ID NO: 18 - protein, 9 aa, Artificial sequence
  Synthetic polypeptide
TSLRGDTTW

SEQ ID NO: 19 - protein, 9 aa, Artificial sequence
  Synthetic polypeptide
PNFRGDEAY

SEQ ID NO: 20 - protein, 9 aa, Artificial sequence
  Synthetic polypeptide
AGVPRGDSP

SEQ ID NO: 21 - protein, 9 aa, Artificial sequence
  Synthetic polypeptide
PRSTRGDST

SEQ ID NO: 22 - protein, 9 aa, Artificial sequence
  Synthetic polypeptide
PFGVRGDDN

SEQ ID NO: 23 - protein, 11 aa, Artificial sequence
  Synthetic polypeptide
GFPFRGDSPAS

SEQ ID NO: 24 - protein, 11 aa, Artificial sequence
  Synthetic polypeptide
PSVRRGDSPAS

SEQ ID NO: 25 - protein, 9 aa, Artificial sequence
  Synthetic polypeptide
PFAVRGDRP

SEQ ID NO: 26 - protein, 9 aa, Artificial sequence
  Synthetic polypeptide
PWPRRGDLP

SEQ ID NO: 27 - protein, 9 aa, Artificial sequence
  Synthetic polypeptide
PSGGRGDSP

SEQ ID NO: 28 - protein, 9 aa, Artificial sequence
  Synthetic polypeptide
VGGRGDVGV

SEQ ID NO: 29 - protein, 9 aa, Artificial sequence
  Synthetic polypeptide
ITSRGDHGE

SEQ ID NO: 30 - protein, 9 aa, Artificial sequence
  Synthetic polypeptide
PPGRGDNGG

SEQ ID NO: 31 - protein, 9 aa, Artificial sequence
  Synthetic polypeptide
PVARGDSGA

SEQ ID NO: 32 - protein, 9 aa, Artificial sequence
  Synthetic polypeptide
STDRGDASA

SEQ ID NO: 33 - protein, 9 aa, Artificial sequence
  Synthetic polypeptide
LNPRGDANT

SEQ ID NO: 34 - protein, 11 aa, Artificial sequence
  Synthetic polypeptide
PSVRRGDSPAS

SEQ ID NO: 35 - protein, 9 aa, Artificial sequence
  Synthetic polypeptide
PTTRGDCPD

SEQ ID NO: 36 - protein, 9 aa, Artificial sequence
  Synthetic polypeptide
PGGRGDSAY

SEQ ID NO: 37 - protein, 9 aa, Artificial sequence
  Synthetic polypeptide
PHDRGDAGV

SEQ ID NO: 38 - protein, 9 aa, Artificial sequence
  Synthetic polypeptide
STDRGDASA

SEQ ID NO: 39 - protein, 9 aa, Artificial sequence
  Synthetic polypeptide
ASGRGDGGV

SEQ ID NO: 40 - protein, 9 aa, Artificial sequence
  Synthetic polypeptide
PASRGDSPP

SEQ ID NO: 41 - protein, 14 aa, Artificial sequence
  Synthetic polypeptide
GSTSGSGKSSEGKG

SEQ ID NO: 42 - protein, 19 aa, Artificial sequence
  Synthetic polypeptide
GSTSGSGKSSEGKGGGGGS

SEQ ID NO: 43 - protein, 14 aa, Artificial sequence
  Synthetic polypeptide
GGGGSGGGGSGGGG

SEQ ID NO: 44 - protein, 20 aa, Artificial sequence
  Synthetic polypeptide
GGGGSGGGGSGGGGSGGGGS

SEQ ID NO: 45 - protein, 15 aa, Artificial sequence
  Synthetic polypeptide
GGGGSGGGGSGGGGS

SEQ ID NO: 46 - protein, 13 aa, Artificial sequence
  Synthetic polypeptide
SCVPLMRCGGCCN

SEQ ID NO: 47 - protein, 14 aa, Artificial sequence
  Synthetic polypeptide
PSCVPLMRCGGCCN

SEQ ID NO: 48 - protein, 9 aa, Artificial sequence
  Synthetic polypeptide
GDLIYRNQK

SEQ ID NO: 49 - protein, 23 aa, Artificial sequence
  Synthetic polypeptide
GGGGGGGGGPSCVPLMRCGGCCN

SEQ ID NO: 50 - protein, 208 aa, Artificial sequence
  Synthetic polypeptide
EVVKAMDVYQRSYCHPIETLVDIFQEYPDEIEYIFKPSCVPLMRCGGCCNDAGLECVPTEESNITMQIMRIKPHQGQHIGEMSFLQHNKCECRPKKDGSTSGSGKSSEGKGEVVKFMDVYQRSYCHPIETLVDIFQEYPDEIEYAFKPSCVPLMRCGGCCNDEGLECVPTEESNITMQIMRAKPHQGQHIGEMSFLQHNKCECRPKKD

SEQ ID NO: 51 - protein, 208 aa, Artificial sequence
  Synthetic polypeptide
EVVKAMDVYQRSYCHPIETLVDIFQEYPDEIEYIFKPSCVPLMRCGGCCNDAGLECVPTEESNITMQIMRIKPYQGHHIGEMSFLQHNKCECRPKKDGSTPGSGKSSEGKGEVVKLMDVYQRSYCHPIETLVDIFQEYPDEIEYAFKPSCVPLMRCGGCCNNEGLECVPTEESNITMQIMRAKPHQGQHVGEMSFLQHNECECRPKKD

SEQ ID NO: 52 - protein, 208 aa, Artificial sequence
  Synthetic polypeptide
EVVKAMDVYQRSYCHPIETLVDIFQEYPDEIEYIFKPSCVPLMRCGGCCNDAGLECVPTEESNITMQIMRIKPYRGHHIGEMSFLQHNKCECRPKKDGSTSGSGKSSEGKGEVVKFMDVYQRSYCHPIETLVDIFQEYPDEIEHAFKPSCVPLMRCGGCCNNEGLECVPTEESNITMQIMRAKPHQGQHIGEMSFLQHNKCECRPKKD

SEQ ID NO: 53 - protein, 208 aa, Artificial sequence
  Synthetic polypeptide
EIVKARDVYQRSYCHPIETLVDILQEYPDEIEYIFKPSCVPLMRCGGCCNDAGLECVPTEESNITMQIMRIKPYQGHHIGEMSFLQHNKCECRPKKDGSTSGSSKSSEGKGEVVKFMDVYQRSYCHPIETLVDIFQEYPDEIEYAFKPSCVPLMRCGGCCNNEGLECVPTEESNITMQIMRAKPHQGQHTGEMSFLQHNKCECRPKKD

SEQ ID NO: 54 - protein, 208 aa, Artificial sequence
  Synthetic polypeptide
EVAKAMDVYQKSYCHPIETLVDILQEYPDEIGYIFKPSCVPLMRCGGCCNDAGLECVPTEESNITMQIMRIKPYQGQHIGEMSFLQHNKCECRPKKDGSTSGSGKSSEGKGEVVKFMDVYQRSYCHPIETLVDIFQEYPDKIEYAFKPSCVPLMRCGGCCNNEGLECVPTEESNITMQITRAKPHQGQHIGEMSFLQHNKCECRPKKD

SEQ ID NO: 55 - protein, 208 aa, Artificial sequence
  Synthetic polypeptide
EVVKAMDVYQRSYCHPIETLVDILQEYPDEIGYIFKPSCVPLMRCGGCCNGAGLECVPTEESNITMQIMRIKPHRGQHIGEMSFLQHNKCECRPKKDGSTSGSGKSSEGKGEVVRFMDVYQRSYCHPIETLVDIFQEYPNEIEYAFKPSCVPLMRCGGCCNNEGLECVPTEESNITMQIMRAKPHQGQHIGEMSFLQHNKCECRPKKD

SEQ ID NO: 56 - protein, 208 aa, Artificial sequence
  Synthetic polypeptide
EAVKAMDVYQRSYCHPIETLVDIFQEYPDEIEYIFKPSCVPLMRCGGCCNDAGLECVPTEESNITMQIMRIKPHRGQHIGEMSFLQHNKCECRPKKDGSTSGSGKSSGGKGEVVKFMDVYQRSYCHPIETLVDVFQEYPDEIEYASEPSCVPLMRCGGCCNHEGLECVPTEESNITMQIMRAKPHQGQHIGEMSFLQHNKCECRPKKD

SEQ ID NO: 57 - protein, 208 aa, Artificial sequence
  Synthetic polypeptide
EVVKAMGVYQRSYCHPIETLVDISQEYPDEIEYIFKPSCVPLMRCGGCCNDAGLECVPTEESNITMQIMRIKPHQGHRIGEMSFLQHDKCECRPKKDGSTSGSGKSSEGKGEVVRFMDVYQRSYCHPIETLVDIFQEYPDEIEYAFKPSCVPLMRCGGCCNNEGLECVPTEESNITMQIVRAKPHQGQHIGEMSFLQHNKCECRPKKD

SEQ ID NO: 58 - protein, 208 aa, Artificial sequence
  Synthetic polypeptide
EVVKAMDVYRRSYCHPVETSVDILQEYPDEIEYIFKPSCVPLMRCGGCCNDAGLECVPTEESNTTMQIMRIKPYRGQHIGEMSFLQHNKCECRPKKDGSTSGSGKSSEGKGEVVKFMDVYQRSYCHPIETLVDIFQEYPDEIEYAFKPSCVSLMRCGGCCNNEGLECVPTEESNITVQIMGAKPHQGQHIGEMSFLQHNKCECRPKKD

SEQ ID NO: 59 - protein, 208 aa, Artificial sequence
  Synthetic polypeptide
EVAKAMDVYQRSYCHPIETLVDILQEYPDEIGYIFKPSCVPLMRCGGCCNDAGLECVPTEESNITMQIMRIKPHQGHRIGEMSFLQHDKCECRPKKDGSTSGSGKSSEGKGEVVKFMDVYQRSYCHPIETLVDIFQEYPDEIEYAFKLPCVPLMRCSGYCNNEGLECVPTEESNITMQIMRAKPHQGQHIGEMSFLQHNKCECRPKKD

SEQ ID NO: 60 - protein, 210 aa, Artificial sequence
  Synthetic polypeptide
EVVKAMDVYQRSYCHPIETLVDIFQEYPDEIEYIFKPSCVPLMRCGGCCNDAGLECVPTEESNITMQIMRIKPHQGQHIGEMSFLQHNKCECRPKKDGSTSGSGKSSEGKGEVVKFMDVYQRSYCHPIETLVDIFQEYPDEIEYAFKPSCVPLMRCGGCCNEEGLECVPTEESNITMQIMRPHDRGDAGVHIGEMSFLQHNKCECRPKKD

SEQ ID NO: 61 - protein, 210 aa, Artificial sequence
  Synthetic polypeptide
EVVKAMDVYQRSYCHPIETLVDIFQEYPDEIEYIFKPSCVPLMRCGGCCNDAGLECVPTEESNITMQIMRIKPHQGQHIGEMSFLQHNKCECRPKKDGSTSGSGKSSEGKGEVVKFMDVYQRSYCHPIETLVDIFQEYPDEIEYAFKPSCVPLMRCGGCCNEEGLECVPTEESNITMQIMRPGGRGDSAYHIGEMSFLQHNKCECRPKKD

SEQ ID NO: 62 - protein, 212 aa, Artificial sequence
  Synthetic polypeptide
EVVKAMDVYQRSYCHPIETLVDIFQEYPDEIEYIFKPSCVPLMRCGGCCNDAGLECVPTEESNITMQIMRIKPHQGQHIGEMSFLQHNKCECRPKKDGSTSGSGKSSEGKGEVVKFMDVYQRSYCHPIETLVDIFQEYPDEIEYAFKPSCVPLMRCGGCCNEEGLECVPTEESNITMQIMRPSVRRGDSPASHIGEMSFLQHNKCECRPKKD

SEQ ID NO: 63 - protein, 210 aa, Artificial sequence
  Synthetic polypeptide
EVVKAMDVYQRSYCHPIETLVDIFQEYPDEIEYIFKPSCVPLMRCGGCCNDAGLECVPTEESNITMQIMRIKPHQGQHIGEMSFLQHNKCECRPKKDGSTSGSGKSSEGKGEVVKFMDVYQRSYCHPIETLVDIFQEYPDEIEYAFKPSCVPLMRCGGCCNEEGLECVPTEESNITMQIMRPASRGDSPPHIGEMSFLQHNKCECRPKKD

SEQ ID NO: 64 - protein, 7 aa, Artificial sequence
  Synthetic polypeptide
IKPHQGQ

SEQ ID NO: 65 - protein, 10 aa, Artificial sequence
  Synthetic polypeptide
CTTHWGFTLC

SEQ ID NO: 66 - protein, 11 aa, Artificial sequence
  Synthetic polypeptide
PSVRRGDSPAS

SEQ ID NO: 67 - protein, 9 aa, Artificial sequence
  Synthetic polypeptide
PTTRGDCPD

SEQ ID NO: 68 - protein, 9 aa, Artificial sequence
  Synthetic polypeptide
PGGRGDSAY

SEQ ID NO: 69 - protein, 9 aa, Artificial sequence
  Synthetic polypeptide
PHDRGDAGV

SEQ ID NO: 70 - protein, 9 aa, Artificial sequence
  Synthetic polypeptide
STDRGDASA

SEQ ID NO: 71 - protein, 9 aa, Artificial sequence
  Synthetic polypeptide
ASGRGDGGV

SEQ ID NO: 72 - protein, 9 aa, Artificial sequence
  Synthetic polypeptide
PASRGDSPP

SEQ ID NO: 73 - protein, 121 aa, Artificial sequence
  Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 74 - protein, 97 aa, Artificial sequence
  Synthetic polypeptide
EVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKD

SEQ ID NO: 75 - protein, 218 aa, Artificial sequence
  Synthetic polypeptide
EVVKAMDVYQRSYCHPIETLVDILQEYPDEIGYIFKPSCVPLMRCGGCCNGAGLECVPTEESNITMQIMRIKPHRGQHIGEMSFLQHNKCECRPKKDGGGGSGGGGSGGGGSGGGGSEVVRFMDVYQRSYCHPIETLVDIFQEYPNEIEYAFKPSCVPLMRCGGCCNNEGLECVPTEESNITMQIMRPSVRRGDSPASHIGEMSFLQHNKCECRPKKD

SEQ ID NO: 76 - protein, 218 aa, Artificial sequence
  Synthetic polypeptide
EVAKAMDVYQKSYCHPIETLVDILQEYPDEIGYIFKPSCVPLMRCGGCCNDAGLECVPTEESNITMQIMRIKPYQGQHIGEMSFLQHNKCECRPKKDGGGGSGGGGSGGGGSGGGGSEVVKFMDVYQRSYCHPIETLVDIFQEYPDKIEYAFKPSCVPLMRCGGCCNNEGLECVPTEESNITMQITRPSVRRGDSPASHIGEMSFLQHNKCECRPKKD

SEQ ID NO: 77 - protein, 218 aa, Artificial sequence
  Synthetic polypeptide
EIVKARDVYQRSYCHPIETLVDILQEYPDEIEYIFKPSCVPLMRCGGCCNDAGLECVPTEESNITMQIMRIKPYQGHHIGEMSFLQHNKCECRPKKDGGGGSGGGGSGGGGSGGGGSEVVKFMDVYQRSYCHPIETLVDIFQEYPDEIEYAFKPSCVPLMRCGGCCNNEGLECVPTEESNITMQIMRPSVRRGDSPASHTGEMSFLQHNKCECRPKKD

SEQ ID NO: 78 - protein, 218 aa, Artificial sequence
  Synthetic polypeptide
EVVKAMDVYQRSYCHPIETLVDILQEYPDEIGYIFKPSCVPLMRCGGCCNGAGLECVPTEESNITMQIMRIKPHRGQHIGEMSFLQHNKCECRPKKDGGGGSGGGGSGGGGSGGGGSEVVRFEDVLRRSSCHPIETLVDIFQEYPNEIEYAFKPSCVPLMRCGGCCNNEGLECVPTEESNITMQIMRPSVRRGDSPASHIGEMSFLQHNKCECRPKKD